(19) United States
(12) Patent Application Publication
(10) Pub. No.: US 2024/0000605 A1
(43) Pub. Date: Jan. 4, 2024

(54) DEFORMABLE THERMAL PACK

(71) Applicant: NYCE INNOVATIONS, LLC., Canton, MA (US)

(72) Inventor: Robert BASCHNAGEL, Garden City, NY (US)

(21) Appl. No.: 18/214,392

(22) Filed: Jun. 26, 2023

Related U.S. Application Data
(63) Continuation of application No. 16/531,871, filed on Aug. 5, 2019, now abandoned.
Application No. 16/531,871 is a continuation-in-part of application No. 16/379,221, filed on Apr. 9, 2019, now Pat. No. 11,179,271.
Application No. 16/379,221 is a continuation-in-part of application No. 16/034,388, filed on Jul. 13, 2018, now Pat. No. 11,192,696.
(60) Provisional application No. 62/604,611, filed on Jul. 14, 2017.

(51) Int. Cl. A61F 7/00; A61F 7/02; A61H 9/00; A47G 9/10; A61F 13/10

(52) U.S. Cl. CPC A61F 7/0085; A61H 2201/1647; A61F 7/007; A61H 9/0092; A47G 9/1036; A61F 13/108; A47G 9/1081; A61F 2007/0011; A61F 2007/0027; A61F 2007/0029; A61F 2007/0039; A61F 2007/0042; A61F 2007/0071; A61F 2007/0031; A61F 2007/0054; A61F 2007/0056; A61F 2007/023; A61H 2205/10; A61F 7/02

(57) ABSTRACT

A thermal pack including: a pouch having a thermal material; covers for covering the pouch. The covers having field portions and first and second bonding edges peripherally outside the first and second field portions. A bendable and deformable support ring formed of sheet metal is disposed between the first and second bonding edges. The pouch is retained between the first and second covers by: at least indirect bonding of a first inner surface of the first bonding edge to a first side surface of the support ring and at least indirect bonding of a second inner surface of the second bonding edge to a second side surface of the support ring; and/or sewing an inner periphery of the first bonding edge to an inner periphery of the second bonding edge and/or an outer periphery of the first bonding edge to an outer periphery of the second bonding edge.

### CROSS REFERENCES TO RELATED APPLICATIONS

The present application is a Continuation-In-Part Application of U.S. application Ser. No. 16/531,871 filed on Aug. 5, 2019, which is a Continuation-In-Part Application of U.S. application Ser. No. 16/379,221 filed on Apr. 9, 2019, which is a Continuation-In-Part Application of U.S. application Ser. No. 16/034,388 filed on Jul. 13, 2018, which claims priority from U.S. Provisional Application No. 62/604,611, filed on Jul. 14, 2017 the entire contents of each of which is incorporated herein by reference.

### BACKGROUND

### Field

The present disclosure relates generally to treatment by applied temperature controlled materials, and, in particular, relates the treatment of pain or soreness, and, in greater particularity, relates to thermal treatment of pain or soreness by applied hot or cold packs.

### Prior Art

Numerous types of ice packs and heat packs use straps for holding the packs to the body.

Further, many devices are shown in issued patents and patent applications publications such as the following: US Patent Application Pub. 2001/0051820 shows thermal treatment packs and the retainers for such. These are highly configurable with the use of cloth strips with fasteners. US Patent Application Pub. 2004/0167456 shows a medical wrap about an ankle using hook and loop. Ice or heat packs may be placed thereunder. US Patent Application Pub. 2004/0244412 shows a pouch for ice bags that is attached to clothing by safety pins. US Patent Application Pub. 2001/0051820 shows a pouch with adhesive tabs for attachment. US Patent Application Pub. 2006/0004427 shows a cylindrical tubular body of flexible material with a temperature retaining material therein that can be used on an arm or leg, but not a shoulder. US Patent Application Pub. 2006/0081000 shows a self-adhesive ice bag device pouch. US Patent Application Pub. 2008/0140166 shows a thermal pack with modules inside. One version is tubular. US Patent Application Pub. 2009/0205106 shows a tubular wrist band with elastic bands on the edges, but it does not provide for heating or cooling but for absorbing water on the wrist. US Patent Application Pub. 2011/0093050 shows a thermal wrap with a pouch for foot use. The wrap is held in place by Velcro straps. U.S. Pat. No. 5,395,399 shows a thermal wrap with a pouch for thermal material and is held in place by an elastic cloth. U.S. Pat. No. 7,060,086 shows a tubular thermal pack for use on hands and legs. U.S. Pat. No. 8,603,151 shows a cooling device for application to body parts having an external thermal source and is held in place by straps. All of these references are incorporated by reference.

Thus, there is a need for a more convenient device to threat pain or soreness.

### SUMMARY

Accordingly, a thermal pack for use with a device for circulating hot or cold fluid is provided. The thermal pack comprising: a body having a cavity; a bendable and deformable support one of attached to or disposed within the body, the support being configured such that the body is deformed into a shape matching a contour of a body part and the body is maintained in the shape while applied to the body part by the support; and tubing having one end in fluid communication with the cavity and another end in fluid communication with the device such that the device circulates a hot or cold fluid through the cavity via the tubing.

The thermal pack can further comprise one or more straps attached to the body for securing the body to the body part of the user.

The body can be configured to be applied to one of a neck of the user, a lower back of the user, an arm or leg of the user, an upper back of the user and a knee of the user.

The support can be provided on a periphery of the body.

The support can be provided interiorly of a periphery of the body.

Also provided is an elastic bandage comprising: a body formed of a typical stretchable material; and one or more bendable and deformable supports disposed at least along a portion of a length of the body, the one or more supports being configured such that the body is deformed into a shape matching a contour of a body part and the body is maintained in the shape while applied to the body part by the one or more supports.

The one or more bendable and deformable supports can be disposed along an entire length of the body.

The one or more bendable and deformable supports can be disposed on a surface of the body. The elastic bandage can further comprise a covering disposed on the surface of the body to cover the one or more bendable and deformable supports.

The body can comprise first and second layers and the one or more bendable and deformable supports are disposed between the first and second layers. The first and second layers can comprise a tubular sleeve.

The body can comprise: a hole configured to accept a thumb of a hand of the user; and one or more straps configured to wrap the body around the hand of the user. The one or more bendable and deformable supports can comprise a single bendable and deformable support disposed around a periphery of the body.

Further provided is a brace for application to a body part, the brace comprising: a deformable material for shielding a portion of a user's body from an impact; one or more bendable and deformable supports disposed at least along a portion of the deformable material, the one or more supports being configured such that the body is deformed into a shape matching a contour of the body part and the body is maintained in the shape while applied to the body part by the one or more supports; and one or more straps for securing the deformable material to the user such that the deformable material is disposed at the portion of the user's body.

The one or more bendable and deformable supports can comprise a single bendable and deformable support disposed around a periphery of the deformable material.

The one or more bendable and deformable supports can be disposed on a surface of the deformable material. The brace can further comprise a covering disposed on the surface of the deformable material to cover the one or more bendable and deformable supports.

Still further provided is a cold/hot bag comprising: a body defining a cavity for holding a hot or cold material, the body having an opening in communication with the cavity for inserting the hot or cold material into the cavity; a cap for covering the opening; and one or more deformable and bendable supports disposed on the body to surround the cavity, the one or more supports being configured such that the body is deformed into a shape matching a contour of a body part and the body is maintained in the shape while applied to the body part by the one or more supports.

The one or more deformable and bendable supports can be disposed along a periphery of the body.

The one or more deformable and bendable supports can extend longitudinally away from the opening.

The cold/hot bag can further comprise one or more securing straps attached to the body for securing the body to the body part of a user.

Still further yet provided is a thermal pack comprising: a body having a cavity containing a thermal source; a bendable and deformable support one of attached to or disposed within the body, the support being configured such that the body is deformed into a shape matching a contour of a body part and the body is maintained in the shape while applied to the body part by the support; and first and second securing straps configured to secure the body to first and second portions of the body part, respectively.

The first portion of the body part can be a torso and the second portion of the body part can be a bicep.

The first portion of the body part can be above a knee and the second portion of the body part can be below the knee.

Still further yet provided is a thermal pack comprising: a body having a cavity containing a thermal source, the body having first and second portion defining a cavity where the first and second portions oppose each other; and a bendable and deformable support one of attached to or disposed within the body, the support being configured such that the body is deformed into a shape matching a contour of a body part and the body is maintained in the shape while applied to the body part by the support; and wherein the body further includes a plurality of connection points connecting opposing portions of the first and second portions to fix the first and second portions together at each of the plurality of connection points.

The support can be provided on a periphery of the body.

Still further yet provided is an electric heating device for application to an body part of a user, the electric heating device comprising: a body having one or more electrical resistance wires for generating heat upon an application of electric current to the one or more electrical resistance wires; a bendable and deformable support one of attached to or disposed within the body, the support being configured such that the body is deformed into a shape matching a contour of a body part and the body is maintained in the shape while applied to the body part by the support; and an electrical input connected to the one or more electrical resistance wires for providing the electric current to the one or more electrical resistance wires.

The body can be configured to be applied to a neck of the user.

The support can be provided on a periphery of the body.

The support can be provided interiorly of a periphery of the body.

Still further yet provided is a pillow comprising: a body having a cavity containing pillow stuffing; and a bendable and deformable support one of attached to or disposed within the body, the support being configured such that the body is deformed into a shape matching a contour of a body part and the body is maintained in the shape while applied to the body part by the support.

The body can be configured to fit around a neck of a user.

The body can have first and second ends and the pillow can further comprise a securing device for securing the first and second ends together when the body is disposed around the neck of the user.

The support can be disposed longitudinally within the pillow stuffing to extend from the first end to the second end.

Still further yet provided is a leg compression device for use with a device for providing compressed fluid, the leg compression device comprising: a body having an inflatable bladder, the inflatable bladder being inflated upon delivery of the compressed fluid to the inflatable bladder; a bendable and deformable support one of attached to or disposed within the body, the support being configured such that the body is deformed into a shape matching a contour of a body part and the body is maintained in the shape while applied to the body part by the support; and tubing having one end in fluid communication with the cavity and another end in fluid communication with the device such that the device provides the compressed fluid to inflatable bladder via the tubing.

The leg compression device can further comprise one or more straps attached to the body for securing the body to the body part of the user.

The body can be configured to be applied to one of a lower leg of the user or an upper and lower leg of the user.

The body can comprise: first and second flaps each extending from the inflatable bladder; and one or more securing straps for securing the body to the body part of the user. The support can be provided on a periphery of each of the first and second flaps of the body.

### BRIEF DESCRIPTION OF THE DRAWINGS

FIG. 1 illustrates a configuration of two thermal packs connected together and used on an ankle region of a user;

FIG. 2 illustrates a thermal pack from the two thermal packs illustrated in FIG. 1;

FIG. 3 illustrates an embodiment of an elongated bendable member for use in the thermal packs;

FIG. 4 illustrates a cup and ball rod component of the elongated bendable member of FIG. 3;

FIG. 5 illustrates a thermal pack employing the elongated bendable member of FIG. 3;

FIG. 6 illustrates another embodiment of a thermal pack;

FIG. 7 illustrates a perspective view of the thermal pack of FIG. 6;

FIG. 8 illustrates a sectional view of the thermal pack of FIG. 7 as taken along line 8-8 in FIG. 7;

FIG. 9 illustrates a configuration of two thermal packs connected together;

FIG. 10 illustrates the two thermal packs of FIG. 9 used on a shoulder region of a user;

FIG. 11 illustrates another embodiment of a thermal pack having a removable bendable elongated wire;

FIG. 12 illustrates another embodiment of a thermal pack having adhesion portions at ends thereof;

FIG. 13 illustrates the thermal pack of FIG. 12 used on a head of an infant

FIG. 14 illustrates thermal packs being connected together where only one connector from one thermal pack lines up with the other thermal pack;

FIG. 15 illustrates a thermal pack having a pouch for use with a separate heating or freezing pack;

FIG. 16 illustrates the thermal pack of FIG. 15 in which the separate heating or freezing pack is being disposed in the pouch;

FIG. 17 illustrates another embodiment of a thermal pack having a slot for removing/inserting a secondary pouch having a thermal material;

FIG. 18 illustrates the thermal pack of FIG. 17 in which the thermal material is being removed from the slot;

FIG. 19 illustrates a thermal pack having an embodiment of support frame;

FIG. 20 illustrates a portion of the support frame of FIG. 19; and

FIG. 21 illustrates a sectional view of the support frame of FIG. 20 as taken along line 21-21 in FIG. 20.

FIG. 22 illustrates a first variation of thermal pack configured for use on the neck and shoulders for connection with a device for circulating hot or cold fluid through the thermal pack.

FIG. 23 illustrates a second variation of thermal pack configured for use on the lower back for connection with a device for circulating hot or cold fluid through the thermal pack.

FIG. 24 illustrates a third variation of thermal pack configured for use on the arm or leg for connection with a device for circulating hot or cold fluid through the thermal pack.

FIG. 25 illustrates a fourth variation of thermal pack configured for use on the upper back for connection with a device for circulating hot or cold fluid through the thermal pack.

FIG. 26 illustrates a fifth variation of thermal pack configured for use on the knee for connection with a device for circulating hot or cold fluid through the thermal pack.

FIG. 27 illustrates a sixth variation of thermal pack configured for use on the lower back for connection with a device for circulating hot or cold fluid through the thermal pack.

FIG. 28a illustrates an ace bandage having deformable wires disposed along a portion of a length of the ace bandage.

FIG. 28b illustrates the ace bandage having deformable flat sheet disposed along the portion of the length of the ace bandage.

FIG. 29a illustrates the ace bandage having deformable wires disposed along an entire length of the ace bandage.

FIG. 29b illustrates an ace bandage having deformable flat sheets disposed along the entire length of the ace bandage.

FIG. 30 illustrates the ace bandage of one of FIGS. 28a or 29b on a wrist of a user.

FIG. 31a illustrates the deformable wires of the ace bandage of one of FIGS. 28a or 29b disposed on an outer surface of the ace bandage.

FIG. 31b illustrates the deformable flat sheets of the ace bandage of one of FIGS. 28b or 29b disposed on the outer surface of the ace bandage.

FIG. 32a illustrates the deformable wires of the ace bandage of one of FIGS. 28a or 29a disposed between two layers forming the ace bandage.

FIG. 32b illustrates the deformable flat sheets of the ace bandage of one of FIGS. 28b or 29b disposed between two layers forming the ace bandage.

FIG. 33 illustrates the deformable wires of FIG. 32a having an end portion configured as shown in FIGS. 20 and 21.

FIG. 34 illustrates a sports brace having deformable wires disposed along a deformable material of the sports brace.

FIG. 35 illustrates the sports brace of FIG. 34 as used on a bicep/elbow of a user.

FIG. 36 illustrates a variation of the sports brace of FIG. 34.

FIG. 37 illustrates the sports brace of FIG. 34 having the deformable wires disposed on a surface of the deformable material.

FIG. 38 illustrates a hand/wrist brace having deformable wires disposed along a deformable material of the hand/wrist brace and applied to the hand/wrist of a user.

FIG. 39 illustrates the hand/wrist brace of FIG. 38 partially applied to the hand/wrist of a user.

FIG. 40 illustrates a first side of the hand/wrist brace of FIG. 38.

FIG. 41 illustrates an opposite side of the hand/wrist brace of FIG. 38.

FIG. 42 illustrates a portion of the hand/wrist brace of FIG. 38 showing the deformable wire.

FIG. 43 illustrates a water/ice bag having one or more deformable wires.

FIG. 44 illustrates a partial view of the deformable water/ice bag of FIG. 44 showing the deformable wire.

FIGS. 45 and 46 illustrate another variation of water/ice bag having deformable wires.

FIGS. 47-49 illustrate another variation of water/ice bag having securing strap and configured for use on a shoulder of a user.

FIGS. 50-52 illustrate a thermal pack for use on the shoulder and having first and second securing straps.

FIGS. 53 and 54 illustrate another variation of thermal pack having securing strap and configured for use on a knee of a user.

FIGS. 55 and 56 illustrate another variation of water/ice bag having securing strap and configured for use on a lower back of a user.

FIGS. 57, 58a and 58b illustrate other variations of thermal pack for use on the upper back and neck of a user.

FIG. 59 illustrates a cyrotherapy machine having a cold fluid pump for circulation through a thermal pack.

FIG. 60a illustrates an exemplary thermal pack for use with the cyrotherapy machine of FIG. 59.

FIG. 60b illustrates another exemplary thermal pack for use with the cyrotherapy machine of FIG. 59.

FIGS. 61 and 62 illustrate an electric heating pad having deformable wires and being configured for the upper back and neck.

FIG. 63 illustrates another variation of electric heating pad having deformable wires.

FIG. 64-70 illustrate a travel neck pillow having deformable wires.

FIG. 71 illustrates a leg compression device having an inflatable bladder for prophylaxis for deep vein thrombosis (DVT) and for use with a DVT system, the leg compression device being wrapped around a lower portion of a patient's leg;

FIGS. 72a and 72b illustrate variations of the leg compression device of FIG. 71 in an unfolded state;

FIG. 73 illustrates a variation of the leg compression device of FIG. 71 where the leg compression device being wrapped around lower and upper portions of a patient's leg; and

FIGS. 74a and 74b illustrate variations of the leg compression device of FIG. 73 in an unfolded state.

Like reference numerals refer to like parts throughout the several views of the drawings.

FIG. 75 illustrates a perspective view of another embodiment of thermal pack.

FIG. 76 illustrates an exploded perspective view of the thermal pack of FIG. 75.

FIG. 77 illustrates a further exploded perspective view of the thermal pack of FIG. 76 in which the gasket is further exploded.

FIG. 78 illustrates the exploded view of the gasket and deformable material of FIG. 77.

FIG. 79 illustrates a close-up sectional view of the exploded gasket and deformable material of FIG. 78.

FIG. 80 illustrates a perspective view of a thermal pack;

FIG. 81 illustrates a sectional view of the thermal pack of FIG. 80 as taken along line 81-81 in FIG. 80;

### DETAILED DESCRIPTION

In general, a thermal treatment pack is applied to the body on an arm or leg or shoulder. A pouch for holding a thermal pack or being the thermal pack itself is held between parallel deformable, bendable rods that remain in that position to which bent. The rods may be on all four sides. The pack is generally rectangularly shaped with rounded corners, but other shapes may be considered. One end may be open and sealed by Velcro® after the appropriate thermal source whether a cooling source or heating source, but the pouch may also hold a hot/cold therapy solution. The pouch may be an ice pack. If the source is the pouch, it is sold as a unit. The pack may come in various sizes for an arm, leg or shoulder. Additional features may be added to the pouch such as handles to aid in applying the pack. In general, the pack is held against the body part, i.e., wrist, and then bent around the wrist forming a “loosely” fitting cuff. This would be also done on the shoulder since the bendable rods will remain bent. The invention is directed at a person normally at rest and not contemplated for a person actively moving. In another embodiment of the present invention, a gel pack being either a cold pack or a heat pack, is fixedly attached to the flexible rod and may be used also to keep foods warm or cold.

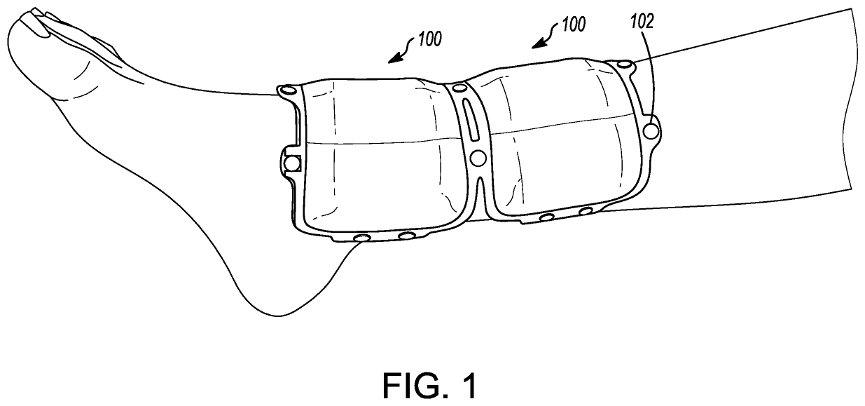

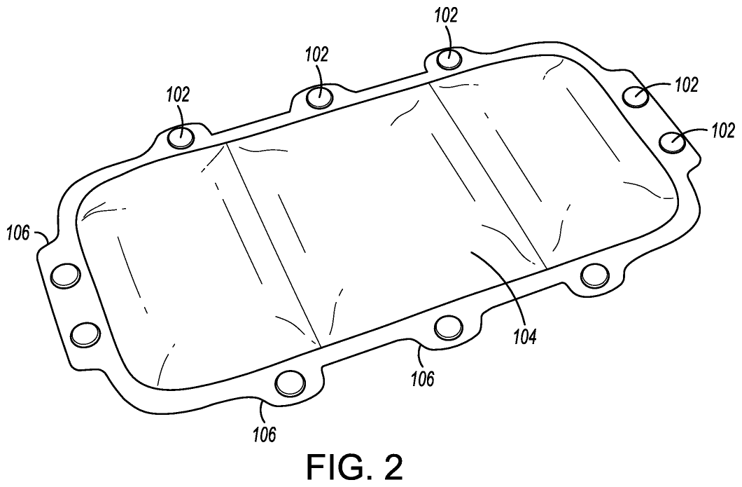

Referring now to FIGS. 1 and 2, there is shown a thermal pack as described above. FIG. 2 shows one such thermal pack 100, while FIG. 1 shows two thermal packs 100 attached together with connectors, such as snaps 102, where the two thermal packs 100 are formed to conform to a shape of an ankle area of a user. The snaps 102 can be configured as those known in the art, such as those used on canvas boat covers, having a male part and a female part which mate together when the male part is pressed into the female part and which can be separated with the application of a force separating the male part from the female part. FIGS. 1 and 2 illustrate the snaps 102 being spaced longitudinally on each of the four sides of the thermal pack 100. The snaps 102 can be providing on only one side, two sides or three sides and can be spaced in any intervals and provided in any number, including a single snap 102. The snaps 102 can be disposed on a pouch projection 106 in a one-to-one correspondence or two or more-to-one correspondence. The snaps 102 can be formed of metal or plastic. Plastic snaps being capable of being used in a microwave where the thermal material in the pouch is being heated in a microwave.

The thermal pack 100 includes a pouch 104 containing a thermal source in a cavity formed by two sides of the pouch 104. The thermal source can be either heated or cooled to apply either hot or cold, respectively, to the ankle area or other body area. Some materials are capable of being both heated and cooled, thereby allowing the thermal pack 100 to be used for application of both heat and cold to a body part of the user. The pouch 104 is formed of materials, such as neoprene and canvas. While all portions of the pouch 104 can be the same material, more than one material can be used to form the pouch 104. For example, one side of the pouch 104 can be a first material, such as neoprene and another side of the pouch 104 can be a second material, such as canvas.

A bendable and deformable support (not shown in FIGS. 1 and 2) that is either attached to or disposed within the pouch 104 is provided such that the pouch 104 can be deformed into a shape matching a contour of a body part and the pouch is maintained in the shape while applied to the body part by the bendable and deformable support frame. Although the bendable and deformable support frame can be provided on all sides of the thermal pack, the bendable and deformable support frame can be providing on only a single side or only on two opposing sides. Still further, the bendable and deformable support frame can be providing interior to the pouch. The bendable and deformable support frame can be a single or plurality of twisted wires formed of a metal material having the desired properties of being capable of being deformed into a shape matching a contour of a body part and allowing the pouch to be maintained in the shape while applied to the body part by the bendable and deformable support frame. The wire or plurality of twisted wires can be encased in an elastomer/resin casing so as to be safe to microwave in the case where the bendable and deformable support frame is fixed to the pouch and the thermal material is heated in the microwave.

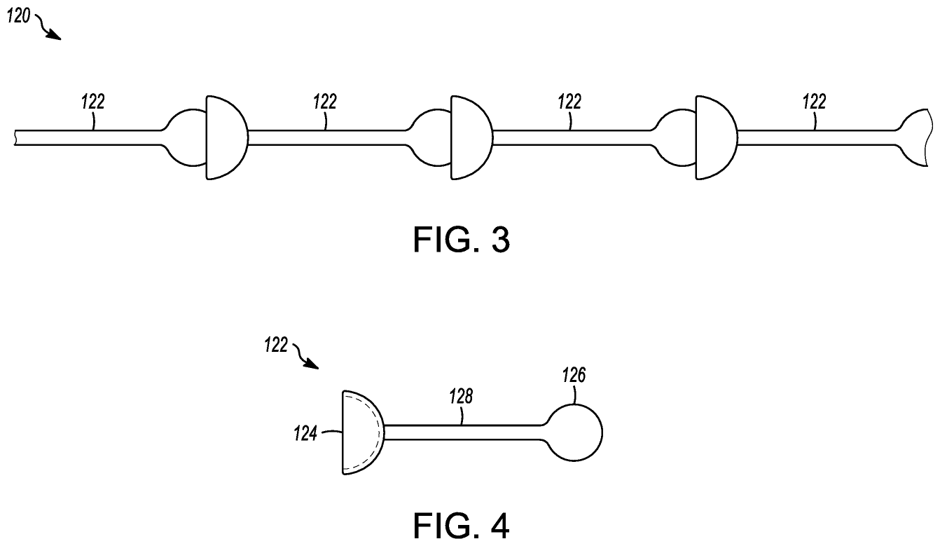

FIGS. 3 and 4 illustrate another embodiment of the bendable support frame in the form of an elongated member 120 having the same properties as a wire in that the elongated member 120 is capable of being deformed into a shape matching a contour of a body part and allowing the pouch to be maintained in the shape while applied to the body part. The elongated member 120 comprises a series of cup and ball members 122. Each cup and ball member 122 includes a cup 124 and ball 126 at each end of a shaft 128. The ball 126 of each cup and ball member 122 fits into a cup 124 of an adjacent cup and ball member 122 such that the ball 126 rotates in the cup 124 in a way to achieve the desired properties discussed above. The ball and cup members 122 can be formed of a resin material and the elongated member 120 can be further covered with a covering, such as a heat shrink tubing, to enhance such properties.

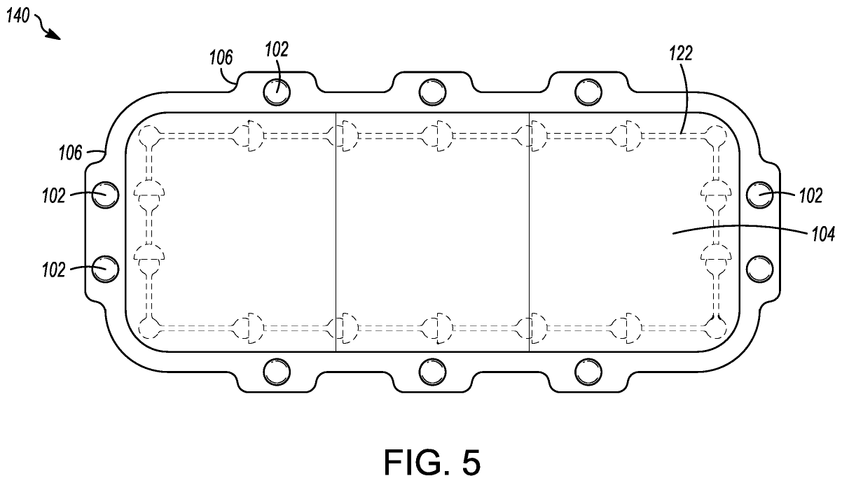

FIG. 5 illustrates an embodiment of thermal pack 140 similar to that of FIG. 2 with the exception that the elongated member 122 is disposed on all four sides of the pouch 104 and within the pouch 104. Alternatively, the elongated member 122 can be provided on less than all four sides and in an interior of the thermal pouch 104. Furthermore, the elongated member 122 can be disposed along an edge of the thermal pack 140 outside of a cavity formed in the pouch 104. Furthermore, the elongated member 122 can be removable from the pouch 104 or thermal pack 140.

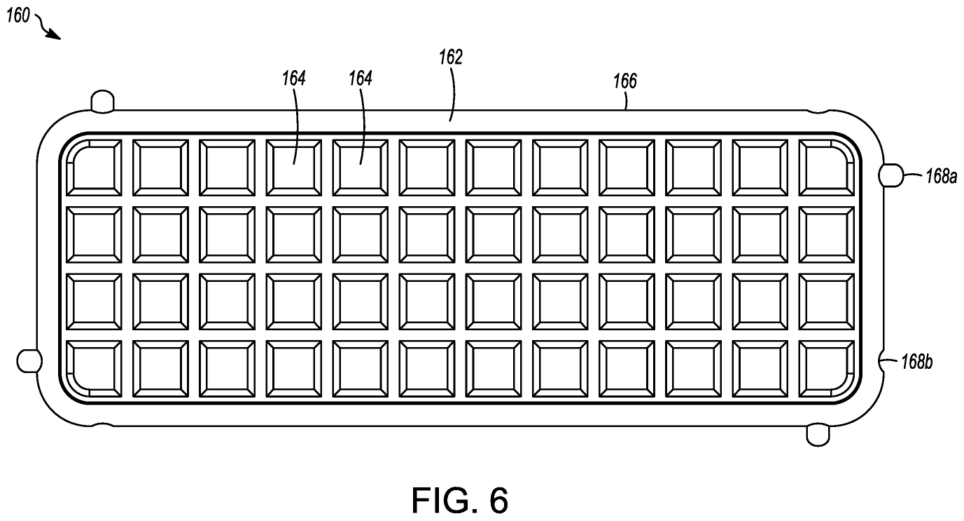

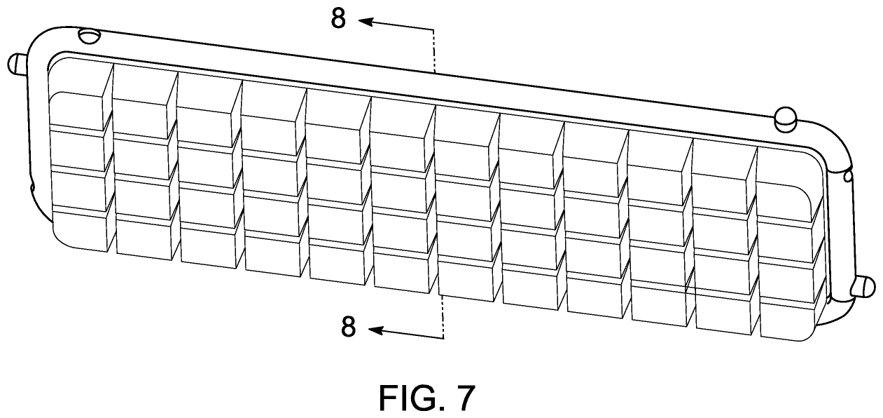

Referring now to FIGS. 6-8, there is shown another embodiment of a thermal pack 160 having a segmented pouch 162 formed of a plurality of pouch segments 164. The pouch segments 164 can be any shape or size and provided in any number greater than two. The thermal pack 160 of FIG. 6 illustrates a great number of small pouch segments 164 for increasing flexibility of the pouch and concentrating an amount of thermal material contained in each pouch segment 164.

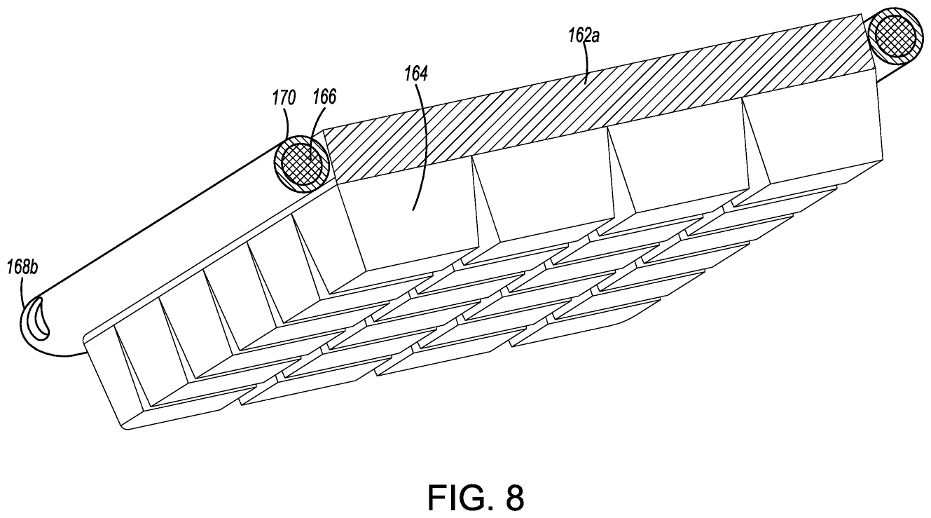

The thermal pack 160 also includes a bendable and deformable support frame 166 provided at all four sides of the pouch 162 to outline the plurality of pouch segments 164. Male and female connectors 168a, 168b are also provided on a periphery of the thermal pack to allow similarly configured thermal packs to be connected together with the thermal pack 160. As shown in FIG. 8 the pouch 162 is formed having the thermal material in both a common portion 162a of the pouch 162 and in each of the pouch segments 164. Furthermore, the bendable and deformable support frame 166 is provided in a cavity 170 apart from the cavity forming the pouch 162.

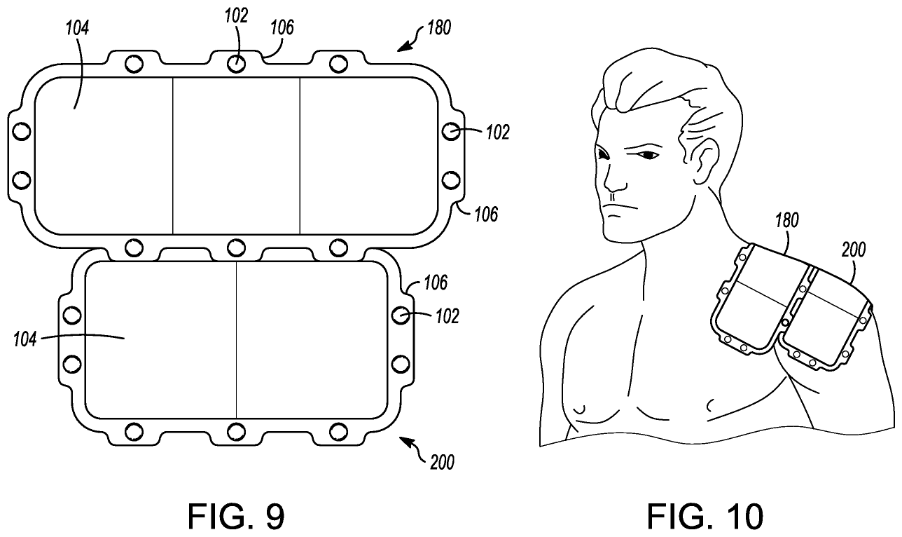

Referring now to FIGS. 9 and 10, there are illustrated first and second thermal packs 180, 200 each having similar features to those described above with regard to any of FIGS. 2, 5 and 7. Thermal pack 180, although larger in size than thermal pack 200 has a similar connector (snaps 102) configuration and spacing such that the larger thermal pack 180 can be connected to the smaller thermal pack 200 to conform with irregular body surfaces, such as the shoulder area illustrated in FIG. 10.

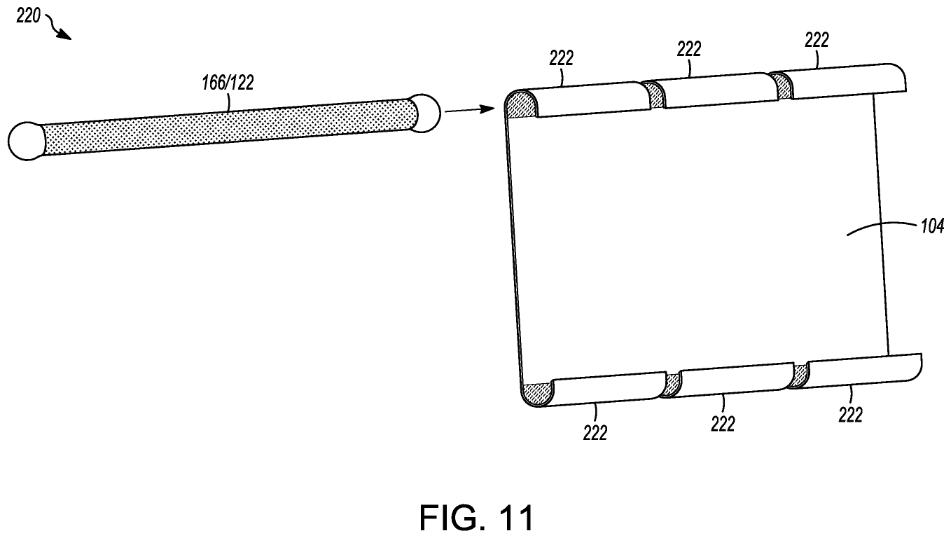

Referring now to FIG. 11, there is shown schematically a thermal pack 220 having similar features to those described above with regard to any of FIGS. 2, 5 and 7. However, in the thermal pack 220 of FIG. 11, the bendable and deformable support frame 166 is removable from the pouch 104. In such configuration, the pouch has ends 222 which can secure the bendable and deformable support frame 166 to the pouch 104. Although a means of releasable securing known in the art can be employed to attach the bendable and deformable support frame 166 to the pouch 104, in the embodiment of FIG. 11, the bendable and deformable support frame 166 has one of a hook or loop fastener (e.g., Velcro™) and the pouch ends 222 has the other of the hook or loop fastener. Such configuration allows use of differently configured bendable and deformable support frame 166 and also allows use of metal components for the bendable and deformable support frame 166 which can be removed when microwaving where the thermal material in the pouch 104 is being heated.

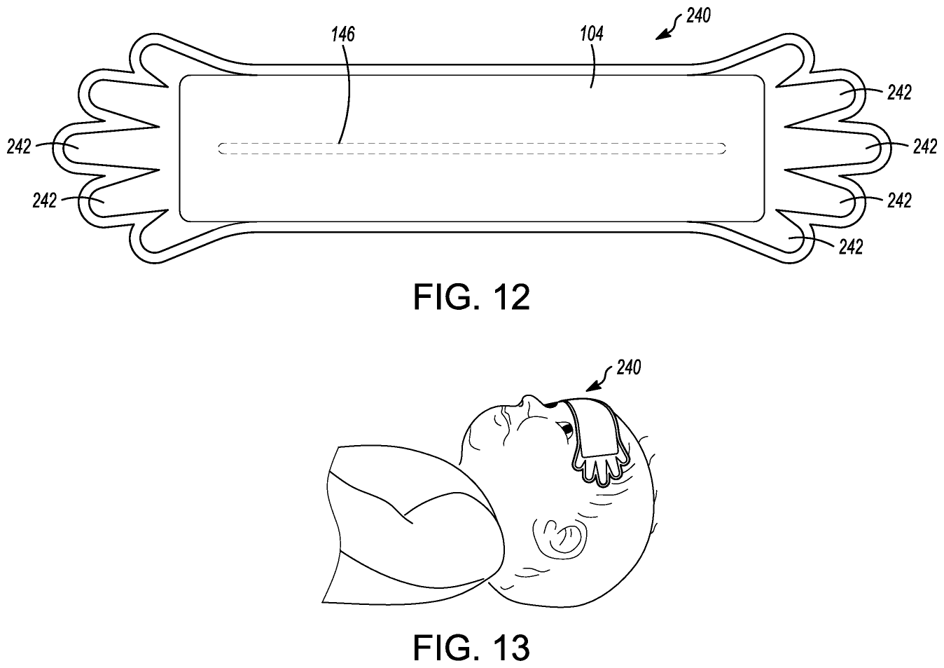

Turning next to FIGS. 12 and 13, there is shown another embodiment of thermal pack 240. The thermal pack 240 of FIGS. 12 and 13 can be configured in any of the ways disclosed in FIGS. 2, 5 and 7. However, in the thermal pack 240 of FIG. 12, the bendable and deformable support frame 166 is centrally located in the pouch 104. Furthermore, although the thermal pack 240 can be configured with connectors 102 for connecting the same to other similarly configured thermal packs, such connectors are not shown in FIG. 12. The thermal pack 240 of FIG. 12 includes one or more projections 242 extending from one or both ends of the thermal pack (FIG. 12 illustrates such projections 242 extending from both ends). The projections 242 include an adhesive for applying the thermal pack to a body part, such as a head of an infant, as shown in FIG. 13. The adhesive can be single or multiple use as is known in the art and can include a backing material that is peeled away to expose the adhesive for use.

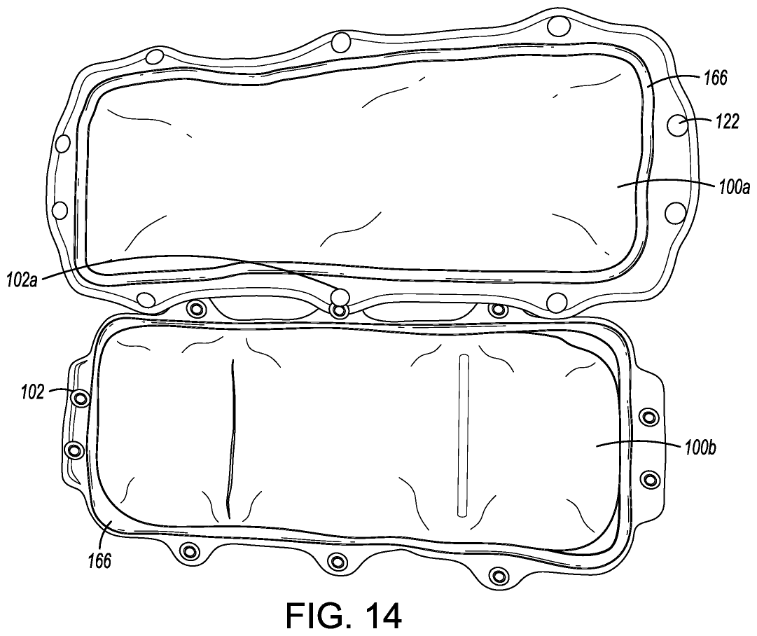

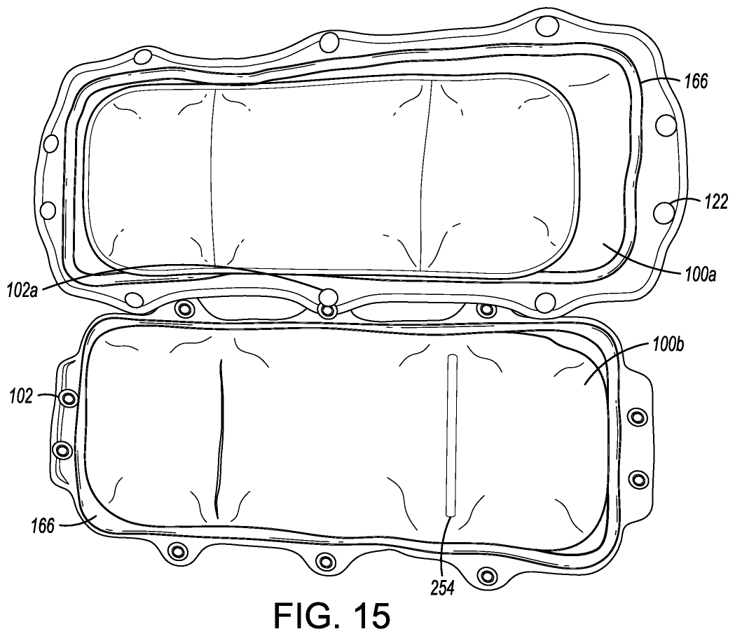

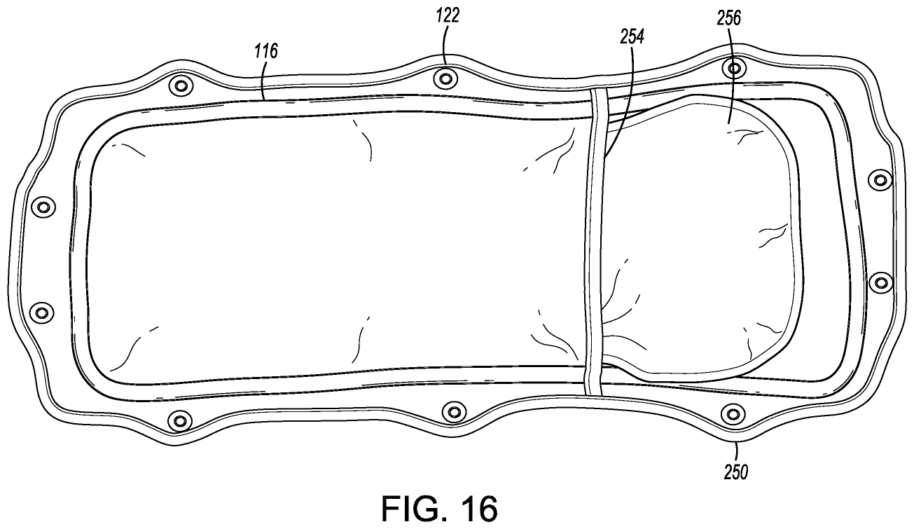

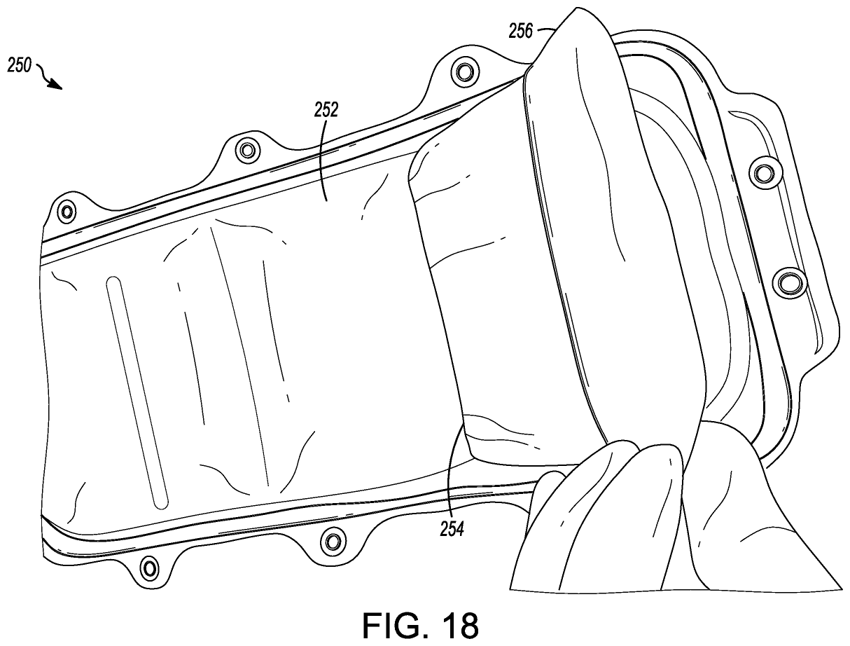

Turning next to FIGS. 14 and 15, there is shown two thermal packs 100a, 100b having the bendable and deformable support frame 166 and connectors 102 misaligned with each other except for a central connector 102a through which the thermal packs 100a and 100b can be connected. Although not optimal, thermal packs can be connected to each other by less than all of the connectors on common sides of the thermal packs, such as by a single connector 102a. In FIG. 15, one of the thermal packs 100b has a slot 254 opening to a pouch described below with regard to FIGS. 16-18.

Turning next to FIGS. 16-18, there is illustrated another embodiment of a thermal pack, generally referred to by reference numeral 250. The thermal pack 250 can be configured as discussed above with regard to any of the above embodiments. However, the pouch 252 (a first pouch) of thermal pack 250 includes a slot 254 for accessing an interior of the pouch 252. The pouch 252 contains a second pouch 256 having the thermal material therein that can be inserted and/or removed through the slot 254. The second pouch 256 can be stored in a freezer or heated in a microwave and then inserted into the pouch 252 while in the cold or heated state.

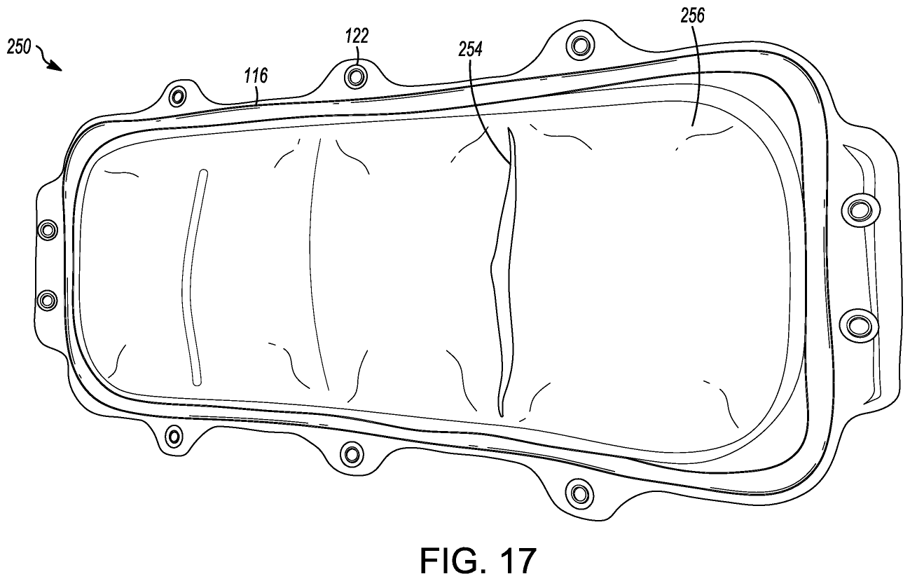

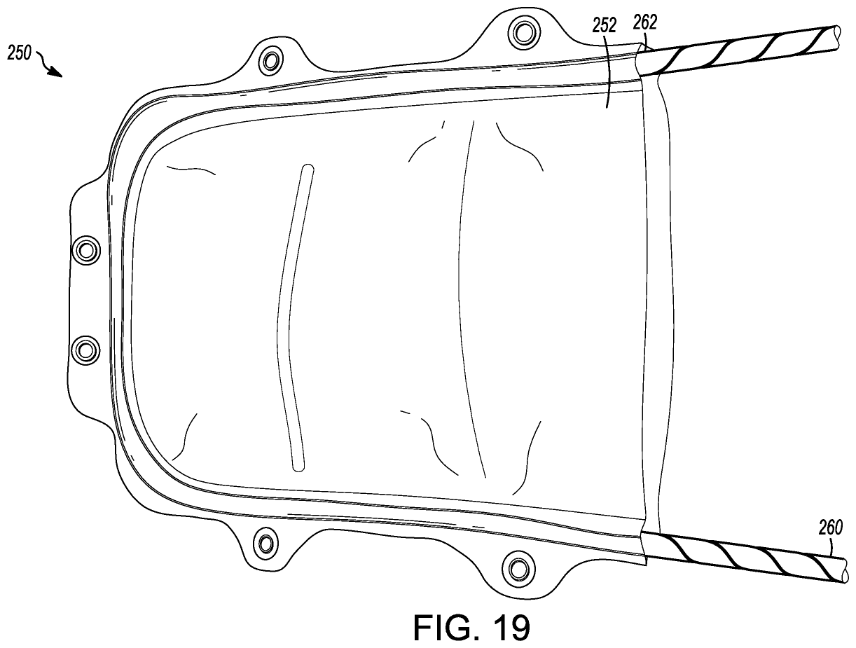

Turning now to FIGS. 19-21, the same illustrate an embodiment of a support frame/wire, generally referred to by reference number 260. FIG. 19 shows the support frame 260 for use with the thermal pack of FIG. 17, however, the support frame 260 can be used with any of the embodiments disclosed above, including that of FIG. 11. Although the support frame 260 is illustrated extending on all four sides of the pouch 252 in FIG. 19, the support frame 260 can extend on one, two or three sides. Furthermore, although the support frame 260 is shown being disposed in a cavity formed along the edges of the thermal pack 250, the support frame 260 can be releasable attached to the thermal pack 250, for example, as shown in FIG. 11.

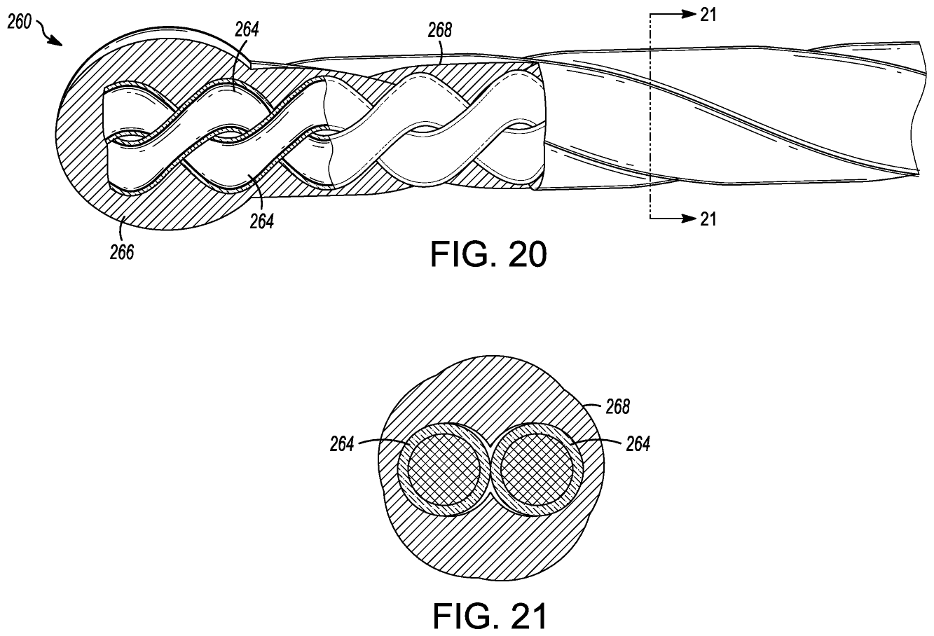

As shown in FIGS. 20 and 21, the support frame/support wire can comprise one wire or a plurality of twisted wires 264 extending along the length of the support frame 260. The wire or plurality of twisted wires 264 are then encased in a resin or elastomer, such as a foam, such as a silicone foam material 268 into a predetermined shape. Such shape can have an elongated shape, as shown in FIG. 20. Such shape can also include a bulb 266 at an end thereof to prevent ends of the wires 264 from puncturing the material of the pouch 252 or cavity 262. Such twisted wires are much less likely to break/kink and they can also be applied to various other products such as pillows, bandages, braces and the like (see below) and can be provided in varying thicknesses based on the particular application. Furthermore, the twisted wires can be made inexpensively for less expensive products including disposable products.

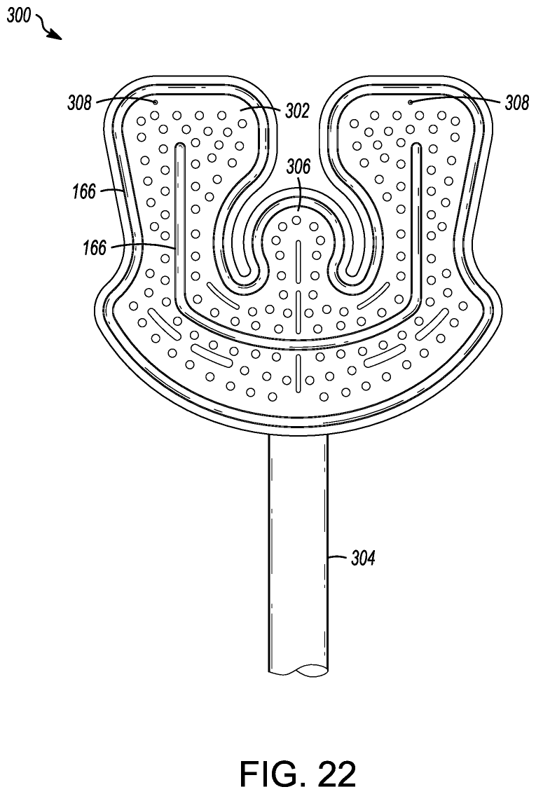

Turning next to FIGS. 22-27, the same illustrate thermal packs for use with a device for circulating hot or cold fluid through the thermal pack, such as cold water. The thermal packs illustrated in FIGS, 22-27 have different configurations for use on different parts of the body, however, such configurations are illustrated by way of example only and such thermal packs can have any configuration, including those discussed above with regard to the thermal packs of FIGS. 1, 2, 5, 6-9 and 12-19.

Specifically with regard to FIG. 22, the same illustrates a thermal pack 300 for use with a device for circulating hot or cold fluids configured for use with the neck of a patient. The thermal pack having a body 302 defining a cavity for holding such hot or cold fluid. The thermal pack 300 further having an inlet/outlet 304 configured to connect to the device for circulating (having for example, a cold or hot source and a pump). The inlet/outlet 304 having connectors (not shown) or the like configured for connection to the device for circulating. The device for circulating may use a separate inlet for providing the hot/cold fluid into the cavity and a separate outlet for withdrawing the hot/cold fluid back to the device or use a single inlet/out for alternately providing and withdrawing the hot/cold fluid so as to maintain an extended period of heat or cold in the thermal pack 300.

The body 302 includes a neck flap portion 306 and shoulder portions 308 configured to fit around a user's neck. The body 302 further including a bendable and deformable support frame 166 (alternatively referred to as a deformable and bendable wire), for example around an outer periphery of the body 302. The bendable and deformable support frame 166 can also be disposed interiorly from the periphery as long as the same does not interfere with the circulation of the hot/cold fluid in the cavity.

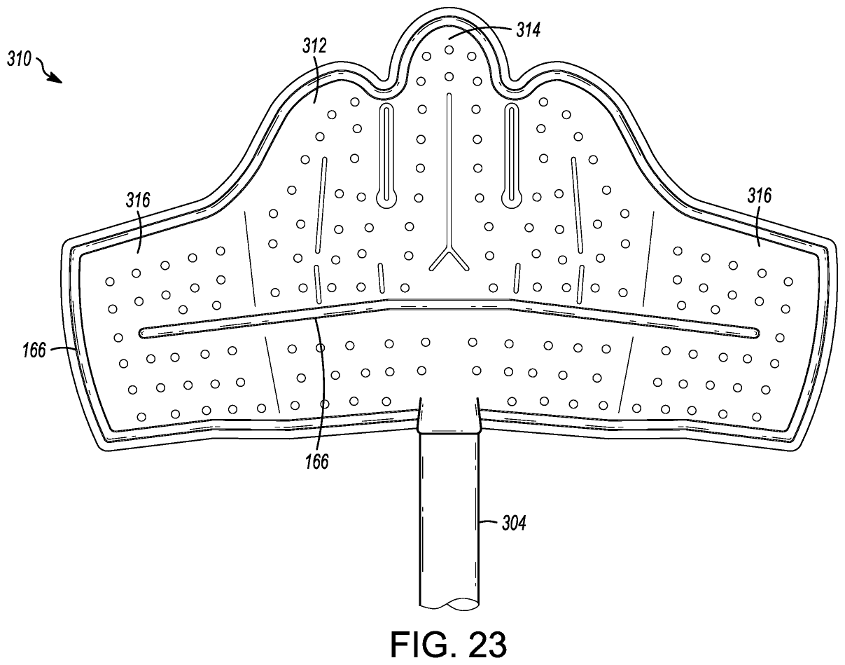

Specifically with regard to FIG. 23, the same illustrates a thermal pack 310 for use with the device for circulating hot or cold fluids configured for use with the lower back of a patient. The thermal pack having a body 312 defining a cavity for holding such hot or cold fluid. The thermal pack 310 further having the inlet/outlet 304 configured as discussed above to connect to the device for circulating.

The body 312 includes a thoracic spine portion 314 and outer waist portions 316 configured to fit a user's lower back. The body 312 further including a bendable and deformable support frame 166, for example around an outer periphery of the body 312. The bendable and deformable support frame 166 can also be disposed interiorly from the periphery as long as the same does not interfere with the circulation of the hot/cold fluid in the cavity.

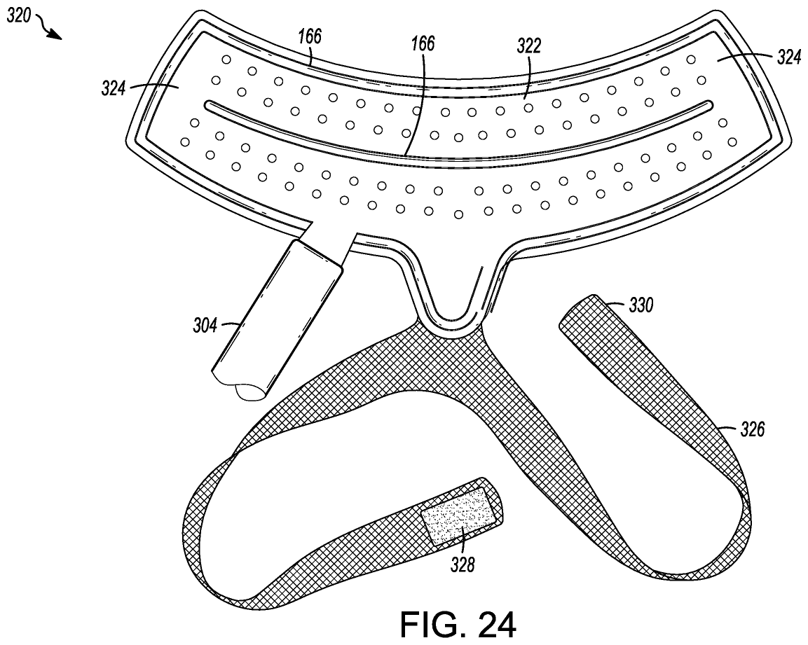

Specifically with regard to FIG. 24, the same illustrates a thermal pack 320 for use with the device for circulating hot or cold fluids configured for use with an arm or a leg of a patient. The thermal pack having a body 322 defining a cavity for holding such hot or cold fluid. The thermal pack 320 further having the inlet/outlet 304 configured as discussed above to connect to the device for circulating.

The body 322 includes elongated portions 324 configured to wrap around a user's arm or leg. The thermal pack 320 may also include a strap 326 to secure the thermal pack around the user's arm or leg, where the strap has a securing means, such as a loop fastener on one end 328 and a mating hook fastener 330 on the other end (commonly referred to by the trade name Velcro®). The body 322 further including a bendable and deformable support frame 166, for example around an outer periphery of the body 322. The bendable and deformable support frame 166 can also be disposed interiorly from the periphery as long as the same does not interfere with the circulation of the hot/cold fluid in the cavity.

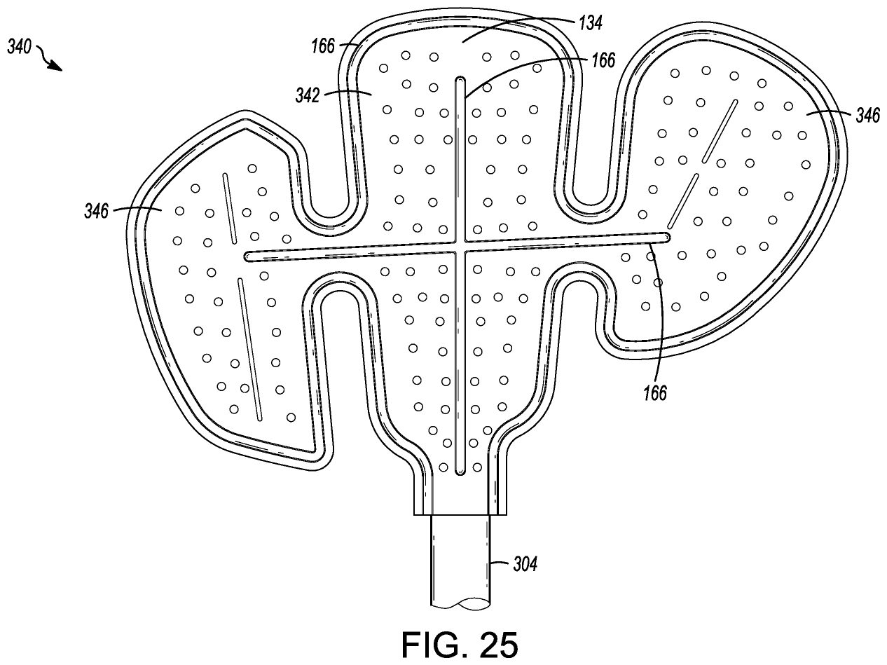

Specifically with regard to FIG. 25, the same illustrates a thermal pack 340 for use with the device for circulating hot or cold fluids configured for use with the upper back/shoulder blades of a patient. The thermal pack having a body 342 defining a cavity for holding such hot or cold fluid. The thermal pack 340 further having the inlet/outlet 304 configured as discussed above to connect to the device for circulating.

The body 342 includes an upper spine portion 344 and outer shoulder blade portions 346 configured to fit a user's upper back. The body 342 further including a bendable and deformable support frame 166, for example around an outer periphery of the body 342. The bendable and deformable support frame 166 can also be disposed interiorly from the periphery as long as the same does not interfere with the circulation of the hot/cold fluid in the cavity.

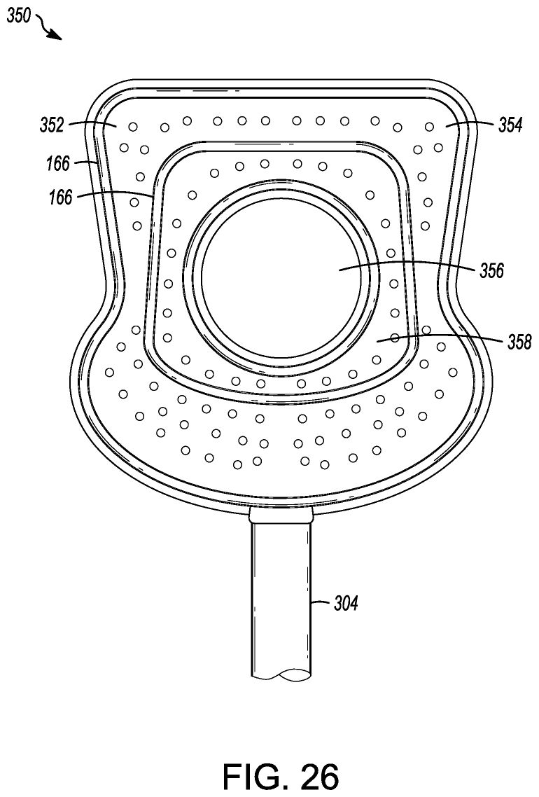

Specifically with regard to FIG. 26, the same illustrates a thermal pack 350 for use with the device for circulating hot or cold fluids configured for use with the knee of a patient. The thermal pack having a body 352 defining a cavity for holding such hot or cold fluid. The thermal pack 350 further having the inlet/outlet 304 configured as discussed above to connect to the device for circulating.

The body 352 includes an outer portion 354 with a knee cap hole 356 configured to fit a user's knee. The body 352 further including a bendable and deformable support frame 166, for example around an outer periphery of the body 352 and an inner periphery of the hole 356. The bendable and deformable support frame 166 can also be disposed interiorly from the periphery as long as the same does not interfere with the circulation of the hot/cold fluid in the cavity unless a portion 358 is not necessary to be heated/cooled by the hot/cold fluid.

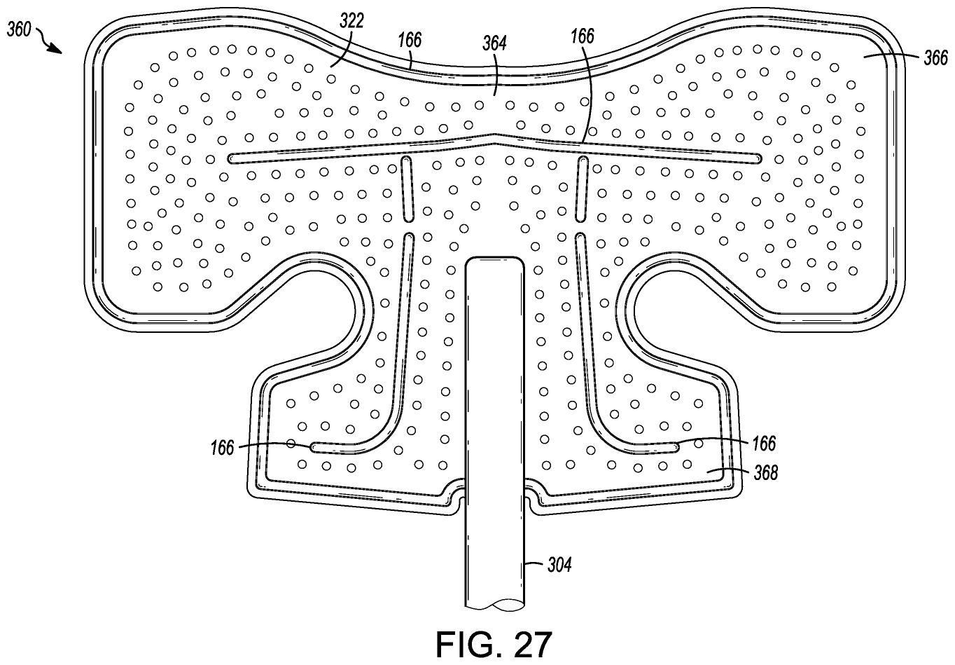

Specifically with regard to FIG. 27, the same illustrates a thermal pack 360 for use with the device for circulating hot or cold fluids having an alternative configuration for use with the lower back of a patient. The thermal pack having a body 362 defining a cavity for holding such hot or cold fluid. The thermal pack 360 further having the inlet/outlet 304 configured as discussed above to connect to the device for circulating.

The body 362 includes a central portion 364 for the thoracic spine, outer waist portions 366 and buttock portions 368 configured to fit a user's lower back. The body 362 further including a bendable and deformable support frame 166, for example around an outer periphery of the body 312. The bendable and deformable support frame 166 can also be disposed interiorly from the periphery as long as the same does not interfere with the circulation of the hot/cold fluid in the cavity.

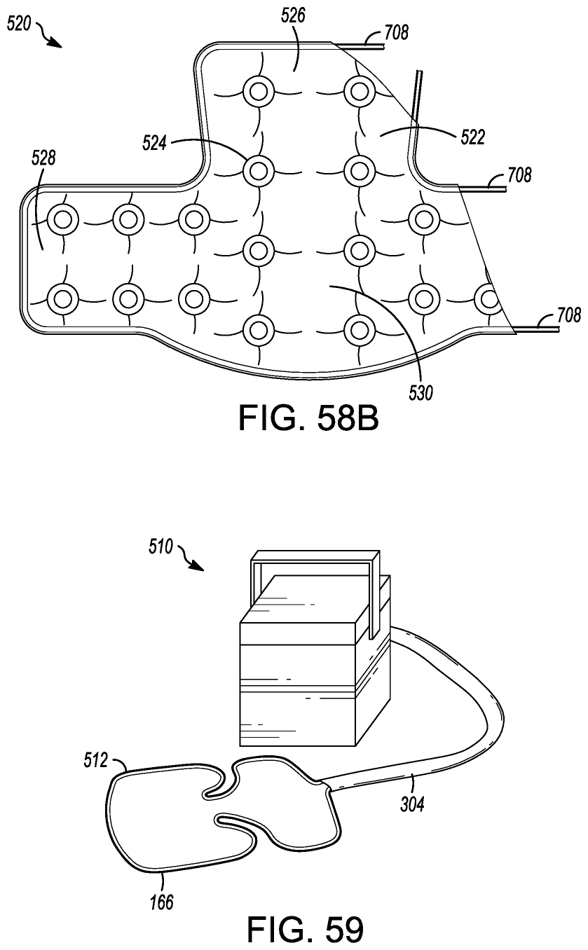

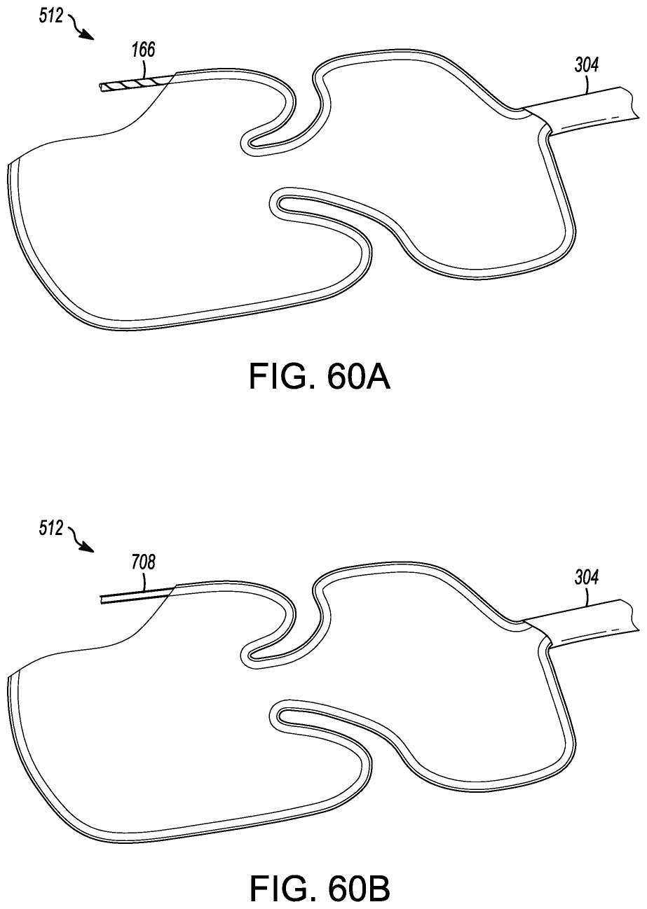

FIG. 59 illustrates a cyrotherapy machine 510 having a hot/cold fluid pump for circulation through the internal cavity of the thermal packs of FIGS. 22-27. The cryotherapy machine 510 having a cold source or heat source and pump for circulating the cold/hot fluid through the thermal packs 300, 310, 320, 340, 350, 360 (shown generically by thermal pack 512 in FIGS. 59 and 60a). The pump circulates the cold/hot fluid to the thermal packs 300, 310, 320, 340, 350, 360, 512 by way of the inlet/outlet tubing 304.

In the thermal packs of FIGS. 22-27 and 60a, the bendable and deformable support frame 166 can have the configuration discussed above with regard to twisted wire 260 (with or without ends 266) and/or elongated member 120.

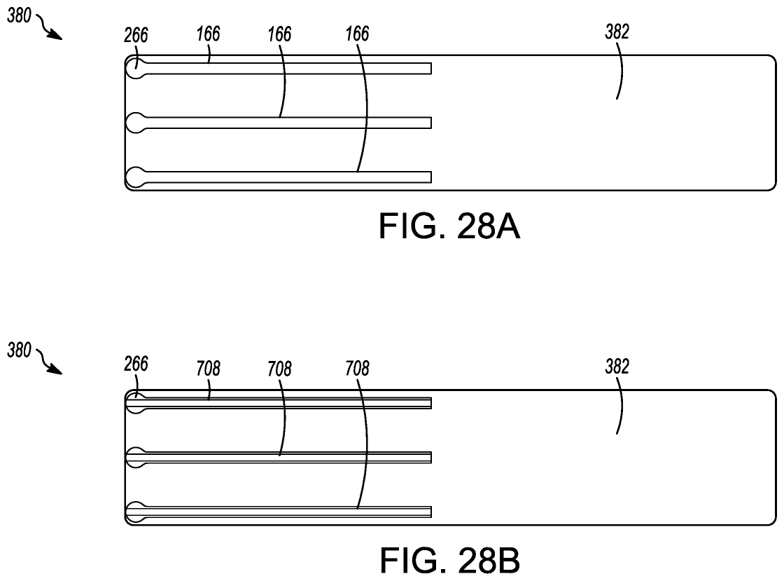

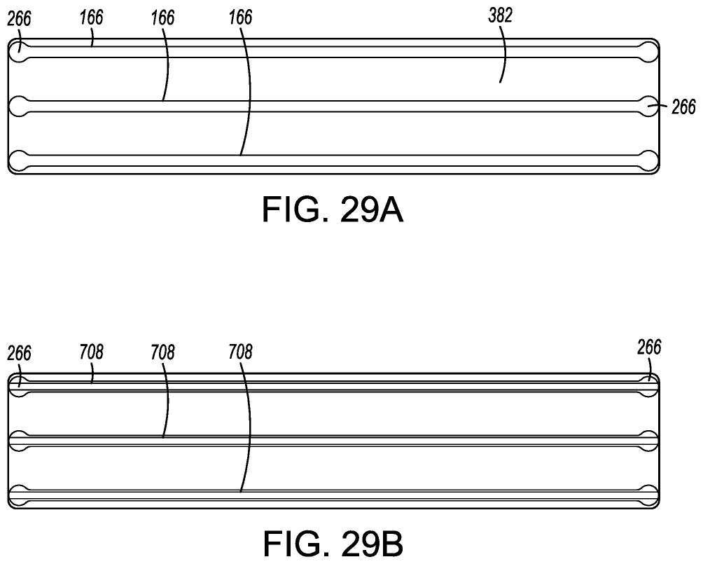

Turning next to FIGS. 28-33 illustrate an elastic bandage or wrap (generally referred to as an ace bandage). In the embodiment of FIG. 28a, the ace bandage 380 has a body 382 formed of a typical stretchable material, such as fabric, where the body 382 has one or more bendable and deformable wires 166 disposed along a portion of a length of the ace bandage 380. FIG. 29a illustrates an embodiment of ace bandage 390 having the deformable wires 166 disposed along an entire length of the ace bandage 390.

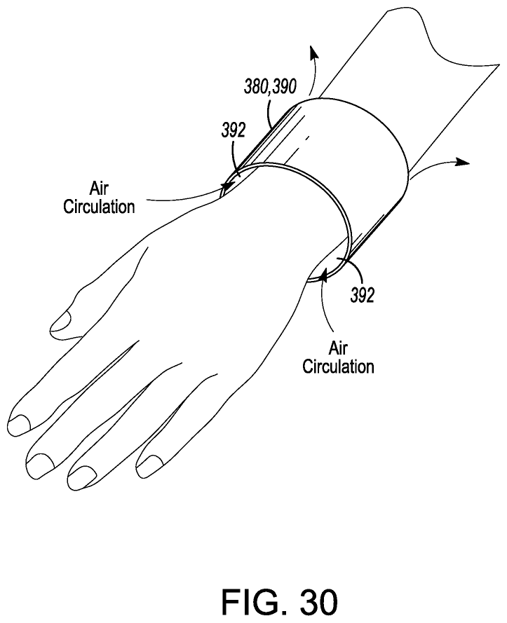

FIG. 30 illustrates the ace bandage of one of FIG. 28 or 29 on a wrist of a user. It is noted that the ace bandage, when applied to the wrist or other appendage, can be “pinched” at 392 to provide a raised portion from the skin, for example, to provide air circulation through the ace bandage while other portions of the ace bandage can fit snugly around the user.

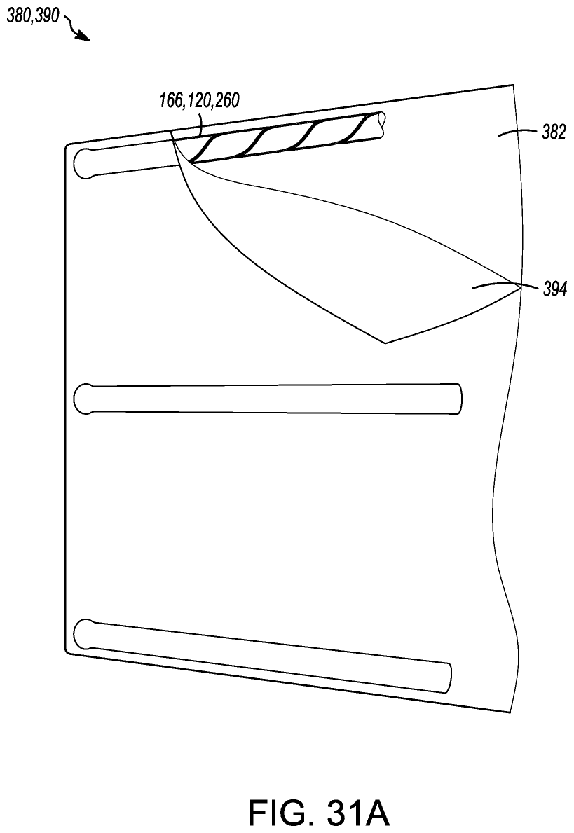

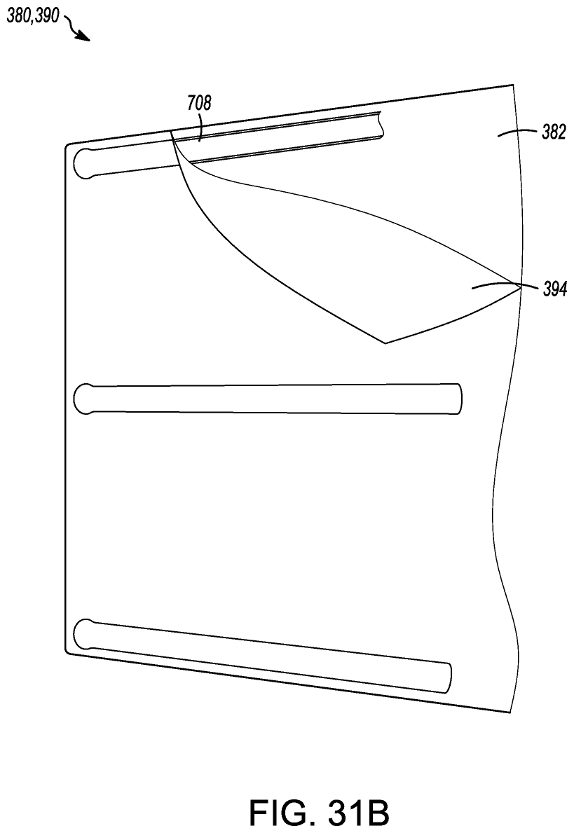

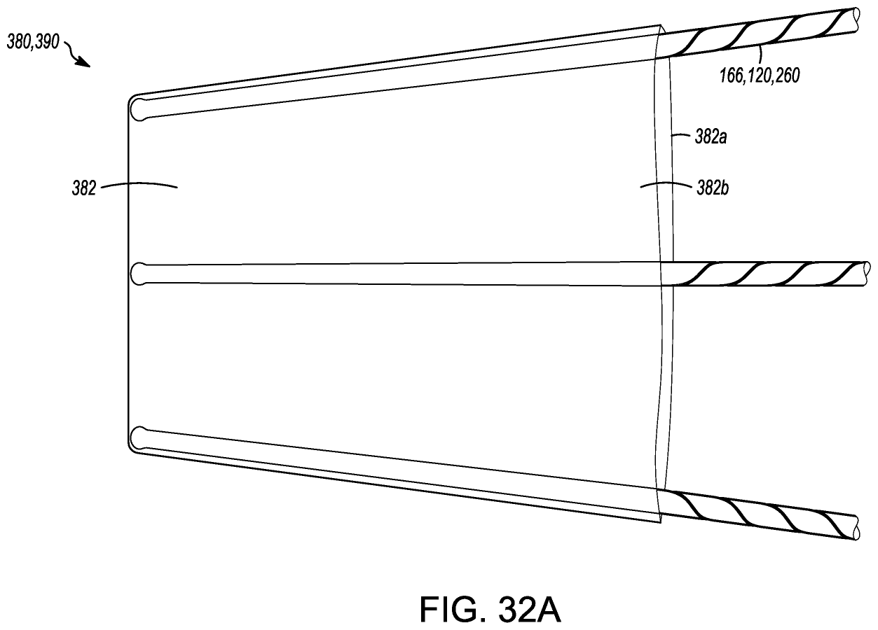

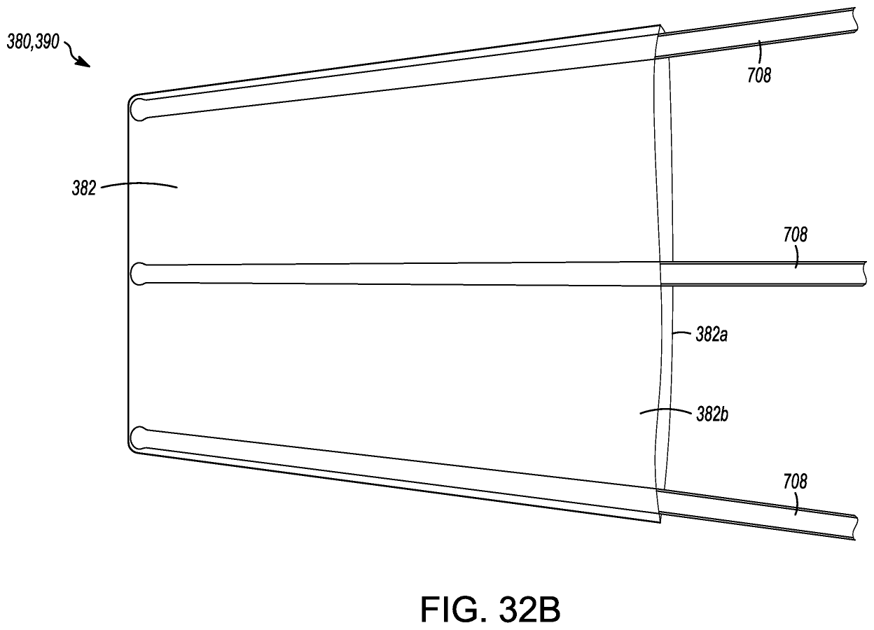

FIG. 31a illustrates the deformable wires 166 of the ace bandage of one of FIGS. 28 or 29 disposed on an outer surface of the ace bandage body 382. In such configuration, and outer cover 394, having similar characteristics to the body 382, can be used to cover the deformable wires 166. Alternatively, as shown in FIG. 32a, the deformable wires 166 of the ace bandage of one of FIG. 28 or 29 can be disposed between two layers 382a, 382b forming the ace bandage body 382. The two layers 382a, 382b can be integrally formed as a tubular sleeve.

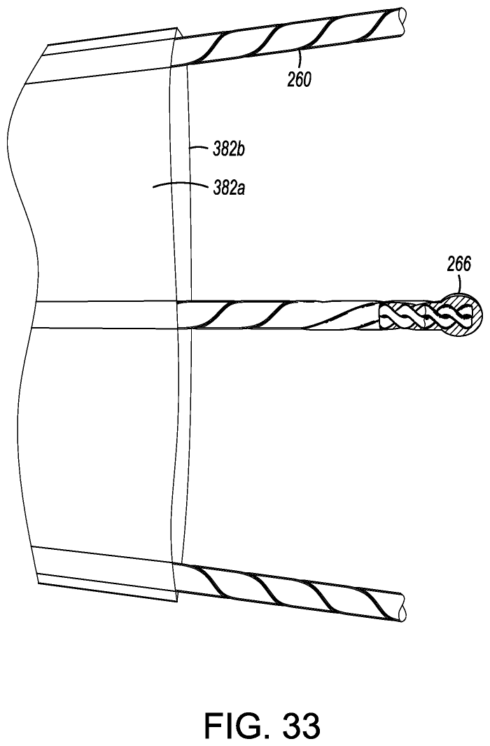

In the ace bandages of FIGS. 28-32, the bendable and deformable support frame 166 can have the configuration discussed above with regard to twisted wire 260 (with or without ends 266) and/or elongated member 120. FIG. 33 illustrates the deformable wires 260 of FIG. 32a having an end portion 266 configured as shown in FIGS. 20 and 21.

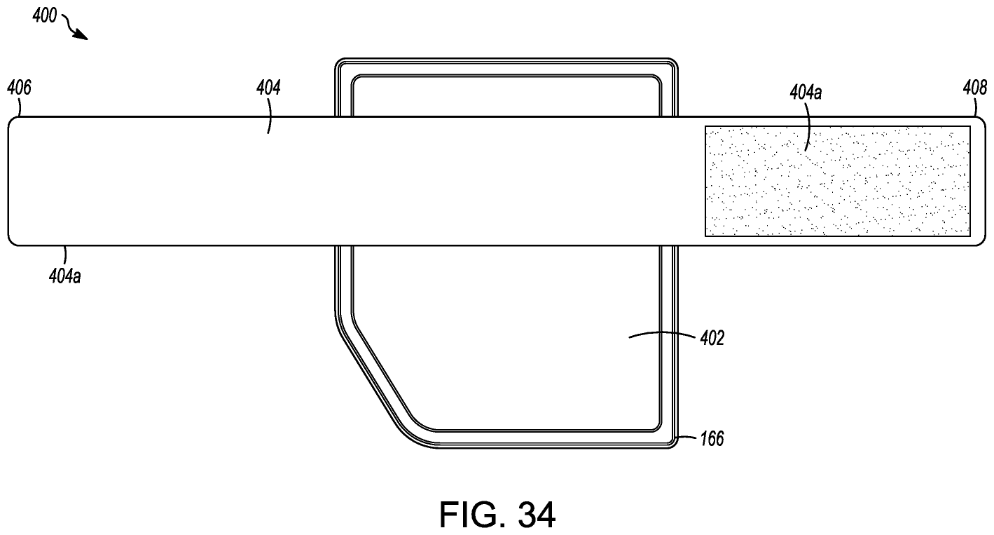

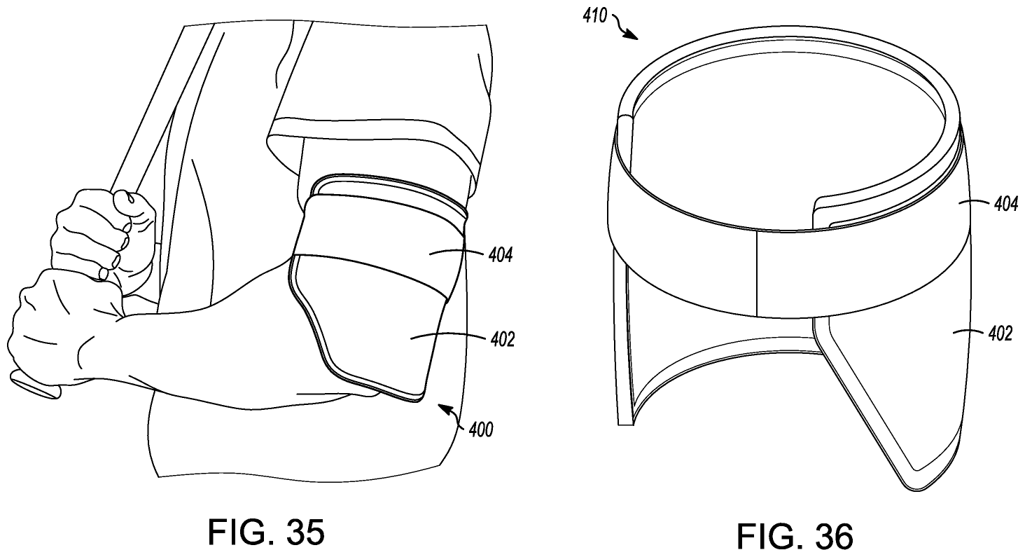

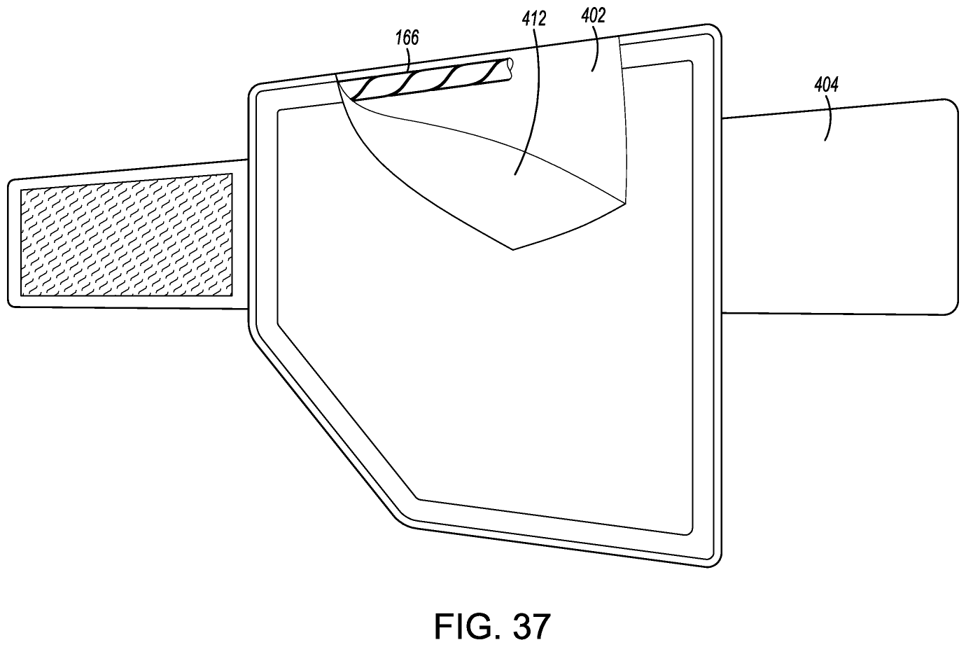

FIGS. 34-37 illustrate a sports brace having deformable wires disposed along a deformable material of the sports brace. FIG. 34 illustrates the sports brace 400 in an unapplied configuration having a deformable brace 402. The deformable brace 402 being formed of a material that is resilient to impact yet can be formed to take the shape of a user's body part being covered and protected. The deformable brace 402 having a bendable and deformable support frame 166 on a periphery or portion thereof such that, once deformed, the deformable brace remains in the deformed configuration. The sports brace further having a strap 404 with a locking means, such as mating portions of a hook and loop fastener 404a on ends 406, 408 thereof. FIG. 35 illustrates the sports brace of FIG. 34 as used on a bicep/elbow of a user to protect the user from impact, such as by a pitched baseball while batting. FIG. 36 illustrates a variation of the sports brace 410 of FIG. 34 having a different strap attachment position. FIG. 37 illustrates the sports brace of FIG. 34 having the deformable wires disposed on a surface of the deformable material 402. In such configuration, and outer cover 412, such as fabric, can be used to cover the deformable wires 166.

In the sports braces of FIGS. 34-37, the bendable and deformable support frame 166 can have the configuration discussed above with regard to twisted wire 260 (with or without ends 266) and/or elongated member 120.

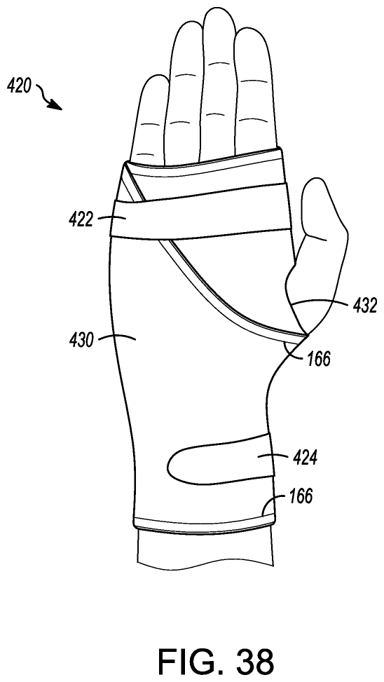

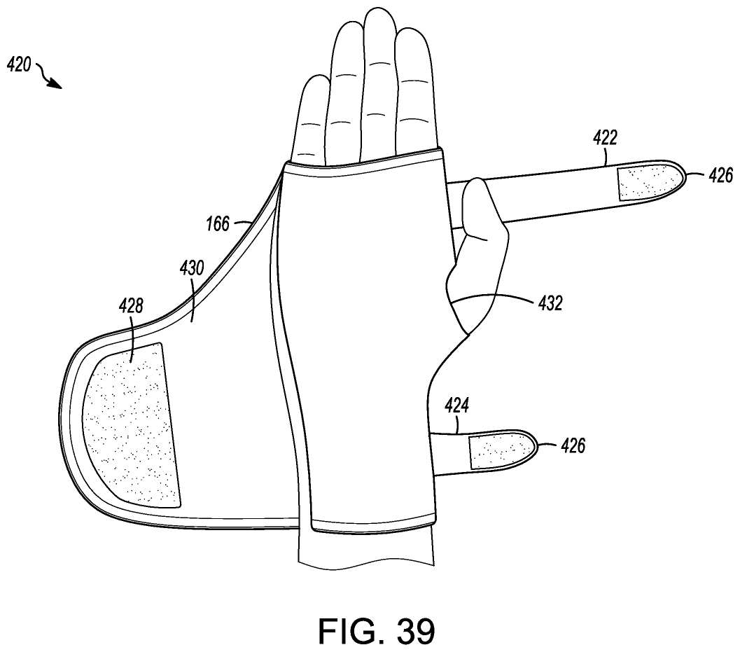

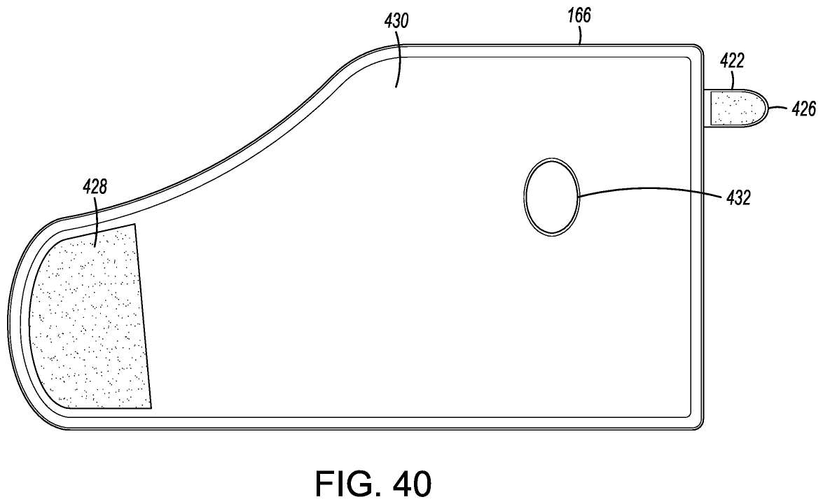

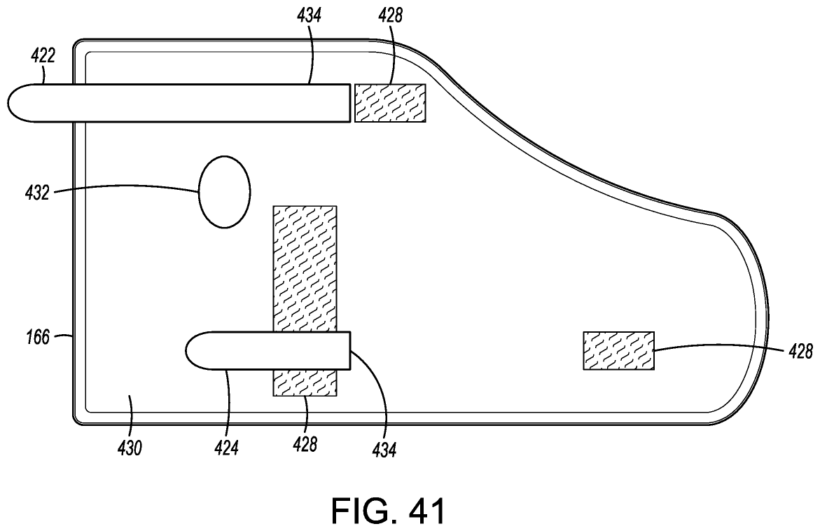

FIGS. 38-42 illustrate an elastic bandage in the form of a hand/wrist brace 420 having deformable wires disposed along a deformable material of the hand/wrist brace and applied to the hand/wrist of a user. FIG. 38 illustrates the hand/wrist brace 420 applied to the wrist and hand of the user while FIG. 39 illustrates the hand/wrist brace of FIG. 38 partially applied to the hand/wrist of the user with straps 422, 424 having fastening means, such as hook and loop fasteners 426, 428. FIG. 40 illustrates a first side of a body 420 of the hand/wrist brace of FIG. 38. The body 430 having the deformable and bendable wire 166 disposed along a periphery of the body 430 (or a portion thereof). The body 430 having a hole 432 corresponding to a thumb of the user's hand. FIG. 41 illustrates an opposite side of body 430 of the hand/wrist brace of FIG. 38. The opposite side of the body 430 having a fixation point 434 for one end of the straps 422, 424 as well as mating hook and loop fasteners 428 for fixation of an other (free) end of the straps 422, 424 having the hook and loop fasteners 426.

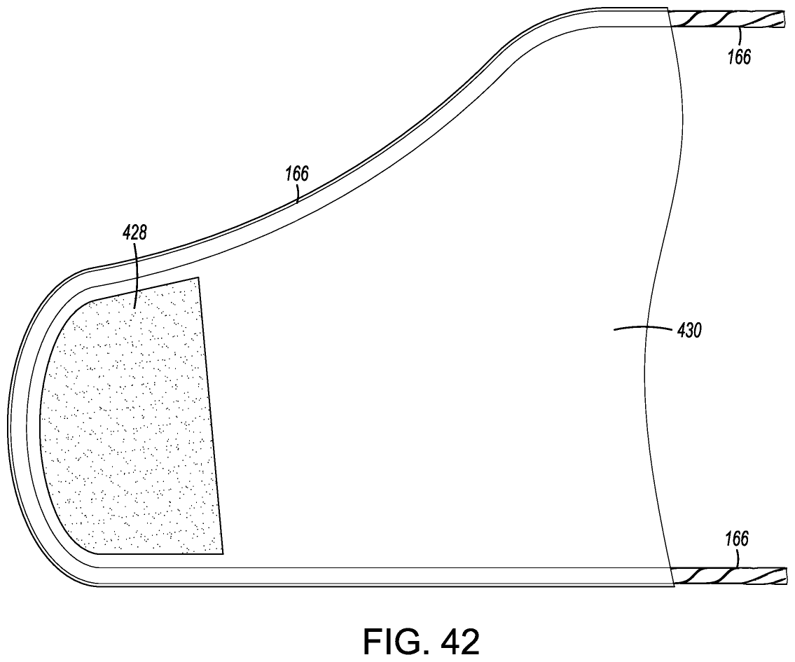

FIG. 42 illustrates a portion of the hand/wrist brace of FIG. 38 showing the deformable wire 166. In the hand/wrist braces of FIGS. 38-42, the bendable and deformable support frame 166 can have the configuration discussed above with regard to twisted wire 260 (with or without ends 266) and/or elongated member 120.

With reference to FIGS. 38 and 39, the deformable wire can be used to create a gap (similar to a pinched portion as discussed above) between the user's wrist and/or hand while allowing the body 430 to fit snugly in other areas, such as around the palm or fingers.

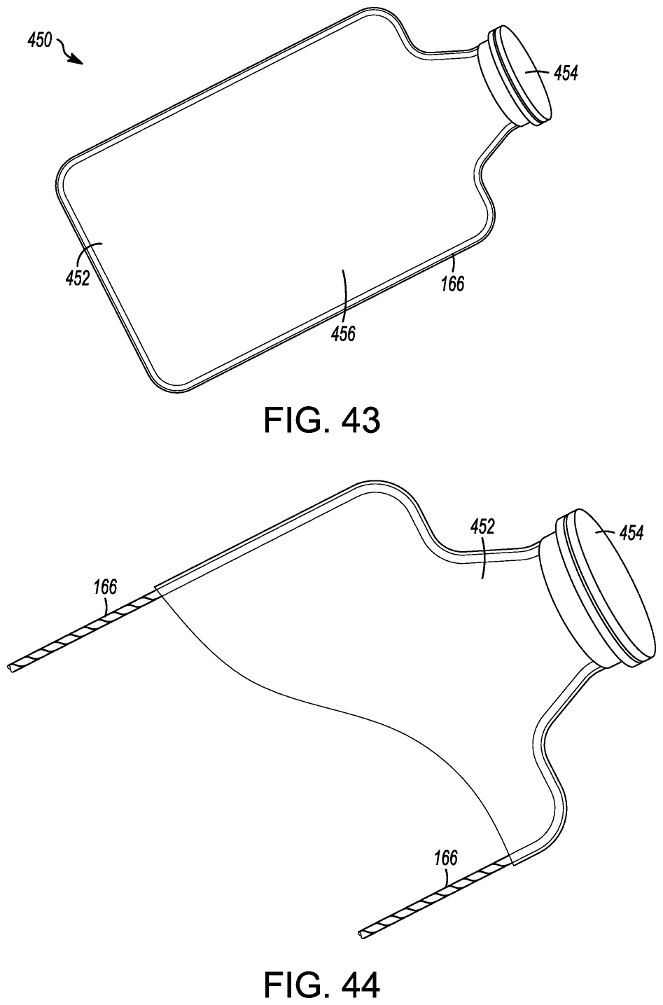

FIGS. 43-49, 55 and 56 illustrate various configurations of water/ice bags having one or more deformable wires disposed in or surrounding a body. FIG. 43 illustrates a first variation of such water bag 450 having the deformable and bending wire 166 disposed around a periphery of a body 452 of the water bag 450. The water bag 450 having an opening with a mating cap 454 disposed on the opening by any means known in the art, such as a male thread on an exterior of the opening and a mating female thread on the inside of the cap 454 to sealingly enclose the opening. The opening provides communication to a cavity 456 for holding ice, cold water or a combination of the same. Other hot or cold liquids and solids can also be contained in the cavity 456. As shown in FIG. 44, the deformable and bendable wire 166 is disposed around a periphery of the cavity 456 except for the opening.

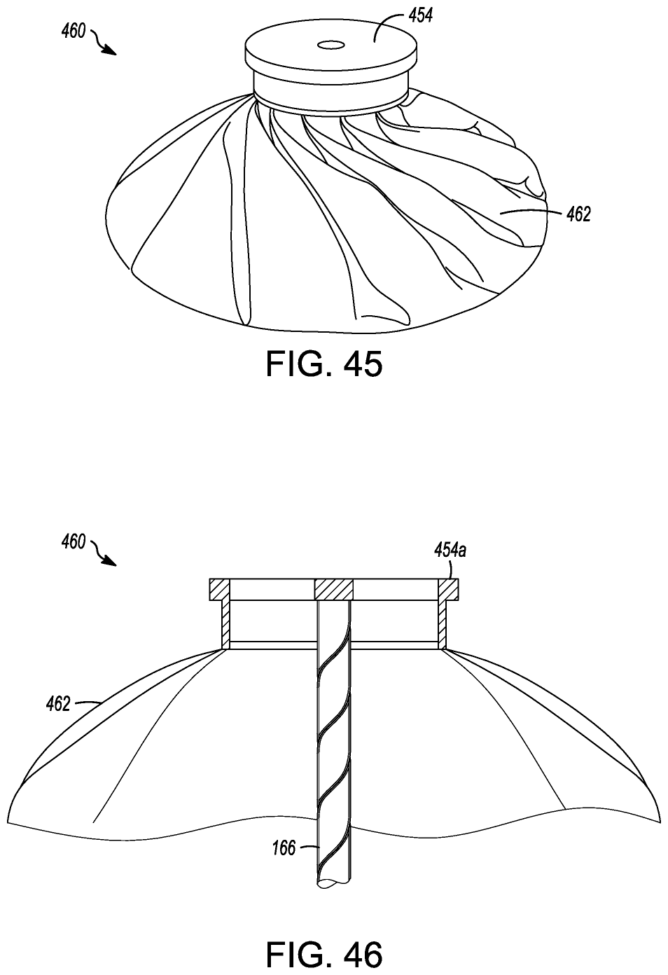

FIGS. 45 and 46 illustrate another variation of water/ice bag 460 having deformable wires 166. Unlike the deformable wire in bag 450, as shown in FIG. 46 (without the cap 454), the deformable wires in water/ice bag 460 can run longitudinally through the body 462 from the opening 454a.

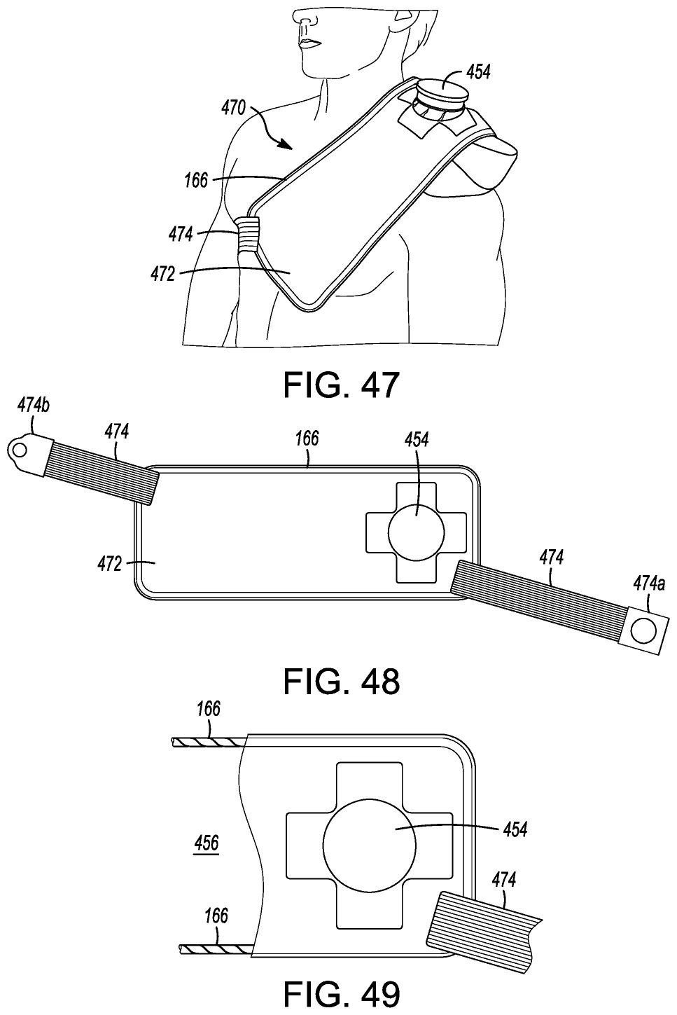

FIGS. 47-49 illustrate yet another variation of water/ice bag 470 having a body 472 with a securing strap 474 attached to the body 472 configured for use on a body part, such as a shoulder of a user. The body 472 has a deformable and bendable wire 166, which can be disposed around a periphery of the cavity 456 of the body 472. In the configuration of FIGS. 47-49, since the opening 454a and cap 454 are provided on a side surface of the body 472, the deformable and bendable wire can be disposed along an entire periphery of the body 472. The strap 474 can have ends 474a, 474b having any securing means known in the art, such as hook and loop fasteners, as described above, or buckles or snaps.

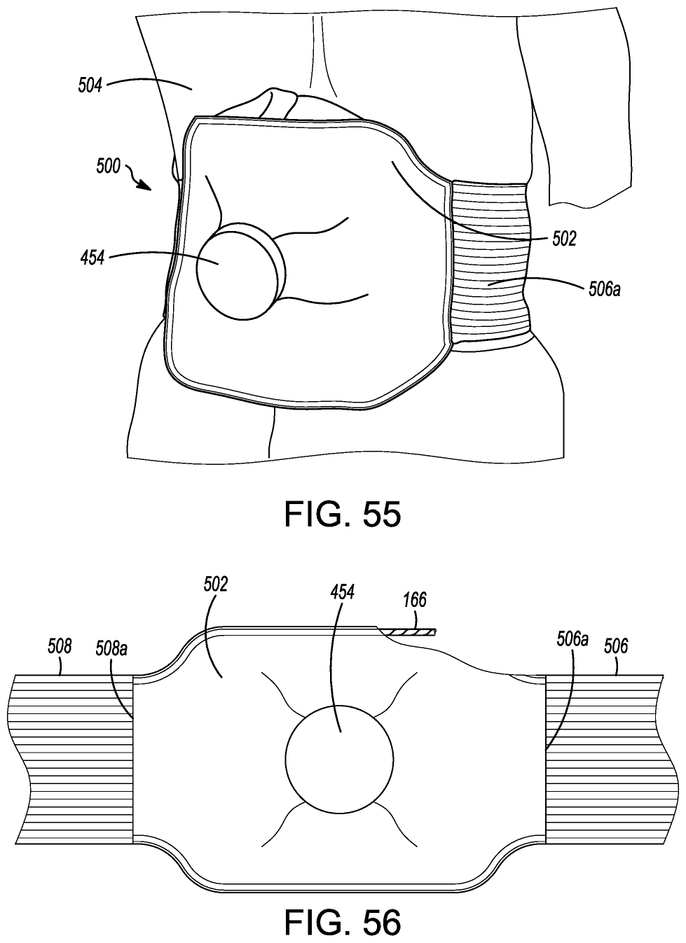

FIGS. 55 and 56 illustrate yet another variation of water/ice bag 500 having a body 502 with a securing strap 504 attached to the body 502 configured for use on a body part, such as the lower back of a user. The body 502 has a deformable and bendable wire 166, which can be disposed around a periphery of the cavity 456 of the body 502. In the configuration of FIGS. 55 and 56, since the opening 454a and cap 454 are provided on a side surface of the body 502, the deformable and bendable wire can be disposed along an entire periphery of the body 502. The strap 504 can have first and second strap pieces, 506, 508 each having a first end 506a, 508a fixed to the body 502 and second ends having any locking means known in the art, such as hook and loop fasteners as described above or buckles. The second end of one strap piece can attach to the second end of the other strap piece, to another portion of the other strap piece or to the body 502. The deformable and bendable wire 166 can be along an entire periphery of the body 502 or a portion thereof, such as all portions of the periphery of the body 502 except where the first ends 506a, 508a are fixed to the body 502.

In the ice/water bags of FIGS. 43-49 and 55-56, the bendable and deformable support frame 166 can have the configuration discussed above with regard to twisted wire 260 (with or without ends 266) and/or elongated member 120.

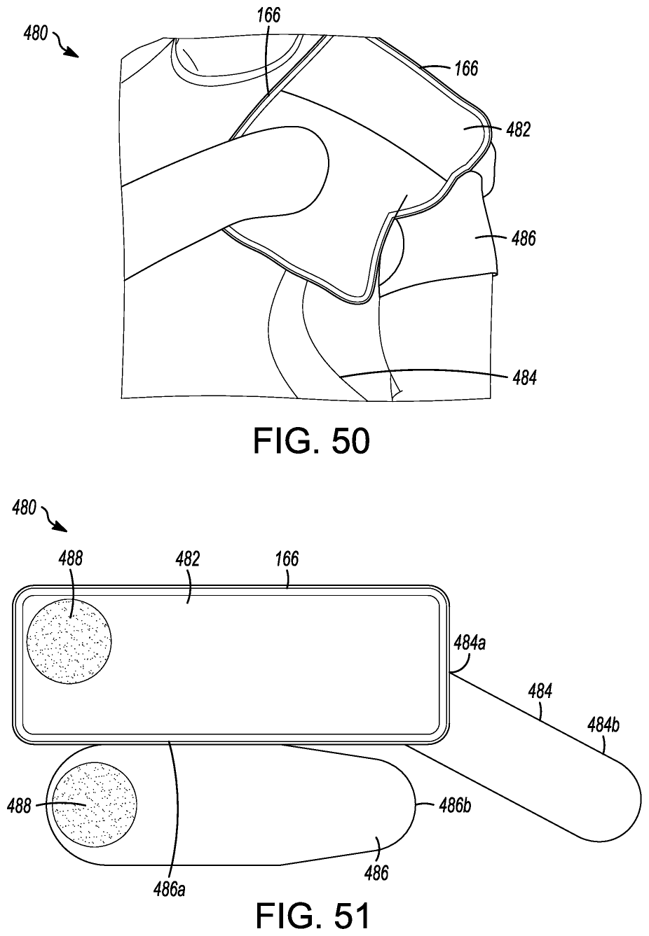

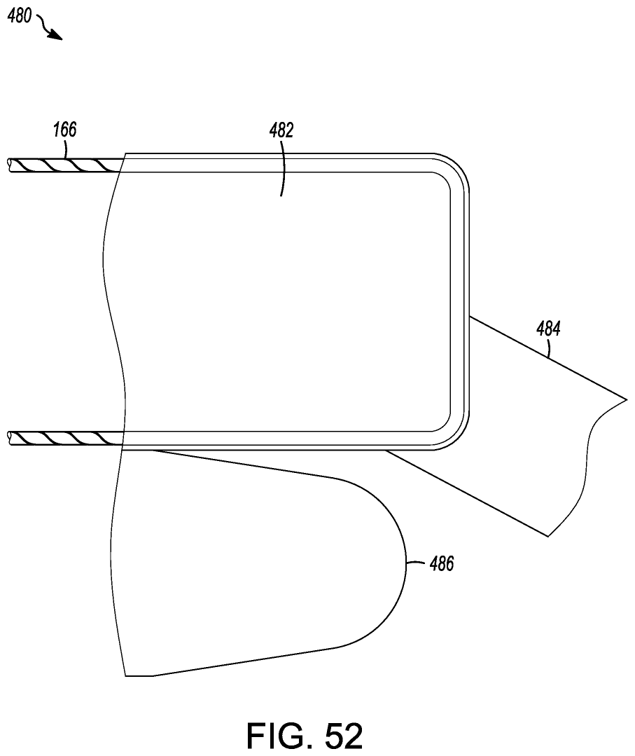

FIGS. 50-52 illustrate a thermal pack 480 having body 482 for holding a hot and/or cold material (either permanently contained in a cavity thereof or removably stored in a cavity through a slot in the body, as discussed above). The body 482 having a deformable and bendable wire 166 around a periphery of the body 482. The body 482 configured for use on a shoulder of a user and having a first securing strap 484 configured to secure around a torso of the user and a second securing strap 486 configured to secure around the upper arm of the user. Each securing strap having a first end 484a, 486a fixed to the body 482 and a second end 484b, 486b having one portion of a locking means, such as a hook and loop fastener. The body 482 has another mating portion of the locking means 488. As with the other straps described throughout this disclosure, the straps can be formed of an elastic material, such as an elastic fabric capable of being stretched in at least a longitudinal direction of the straps.

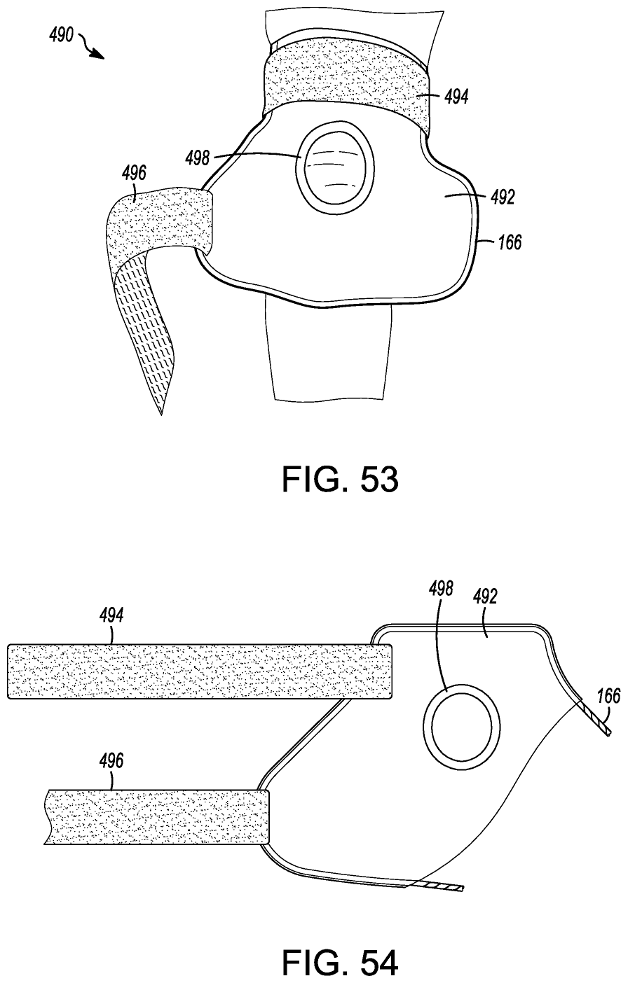

FIGS. 53 and 54 illustrate another variation of thermal pack 490 having a body 492 with first and second securing straps 494, 496 configured to secure the thermal pack 490 to a knee of a user. The body 492 being configured to hold a hot and/or cold material (either permanently contained in a cavity thereof or removably stored in a cavity through a slot in the body, as discussed above). The body 492 having a deformable and bendable wire 166 around a periphery of the body 482. The body 492 having a hole 498 configured for use on a knee of a user and having a first securing strap 494 configured to secure around an upper portion of the knee of the user and a second securing strap 496 configured to secure around a lower portion of the knee of the user. Each securing strap 494, 496 having a first end fixed to the body 492 and a second end having one portion of a locking means, such as a hook and loop fastener. The body 492 has another mating portion of the locking means.

In the thermal packs of FIGS. 50-54, the bendable and deformable support frame 166 can have the configuration discussed above with regard to twisted wire 260 (with or without ends 266) and/or elongated member 120.

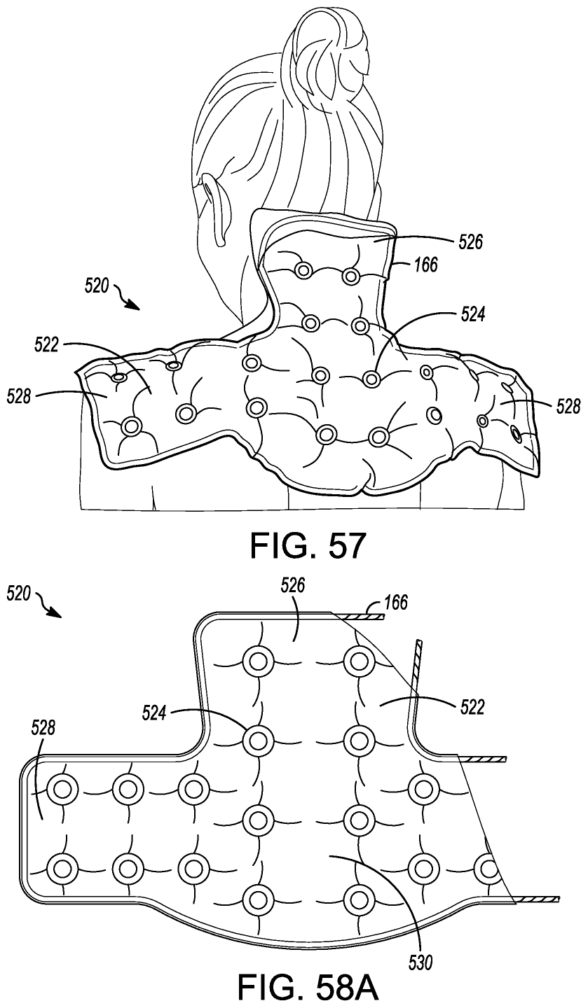

FIGS. 57 and 58a illustrate another variation of thermal pack 520 for use on the upper back and neck of a user. The thermal pack has a tufted body 522 for providing additional flexibility to conform to the shape of the user's body part, such as the neck and upper back. The tufted body 522 having front and back opposing sides defining a cavity for holding the thermal (hot/cold) material. Connection points 524 are formed connecting the front side and back side of the body 522. The body 522 having a neck portion 526 and upper back/shoulder portions 528 extending from a central portion 530. The deformable and bendable wire 166 can extend around portions of the body or through the body, such as around an entire periphery of the body 522. Such bendable and deformable support frame 166 can have the configuration discussed above with regard to twisted wire 260 (with or without ends 266) and/or elongated member 120.

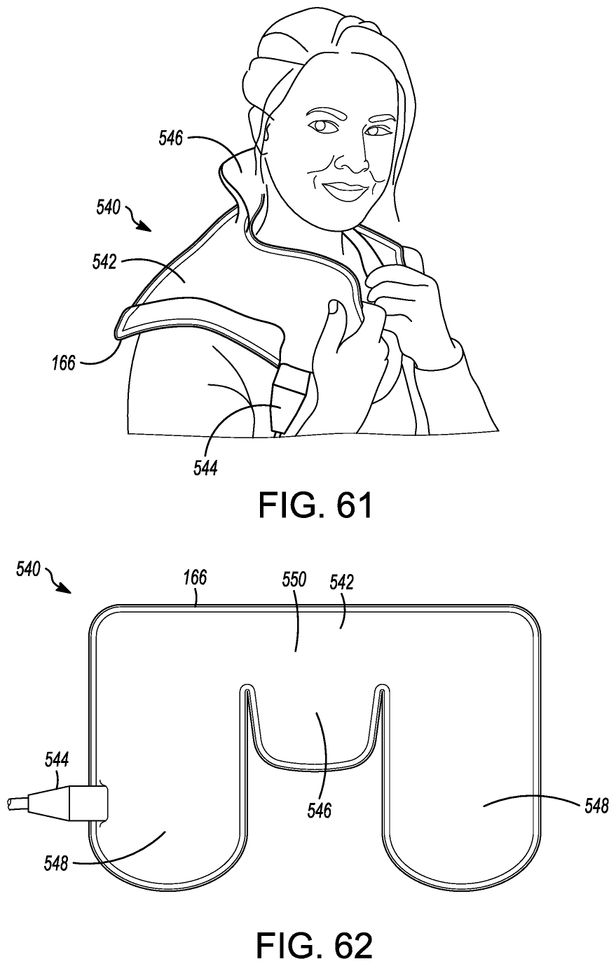

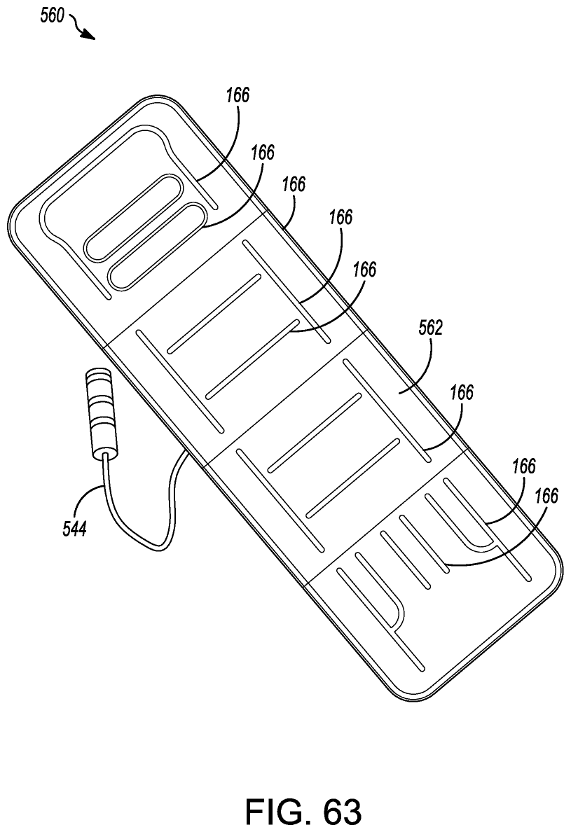

FIGS. 61 and 62 illustrate an electric heating pad 540 configured for the upper back and neck. The electric heating pad 540 having a body 542 configured to produce heat as known in the art. For example, the body 542 having resistance wiring spread through an intended heated portion of the body 542 which are supplied electric current by cable 544. The body 542 having a neck portion 546 and shoulder portions 548 extending from a central portion 550. The deformable and bendable wire 166 can extend around portions of the body or through the body, such as around an entire periphery of the body 542.

FIG. 63 illustrates another variation of electric heating pad 560 having deformable wires 166 disposed around a generically shaped body 562. The body 562 of the electric heating pad of FIG. 63 also includes the bendable and deformable wires 166 disposed within the periphery of the body 562 to provide additional capability to deform to an applied body portion.

In the electric heating pads of FIGS. 61-63, the bendable and deformable support frame 166 can have the configuration discussed above with regard to twisted wire 260 (with or without ends 266) and/or elongated member 120.

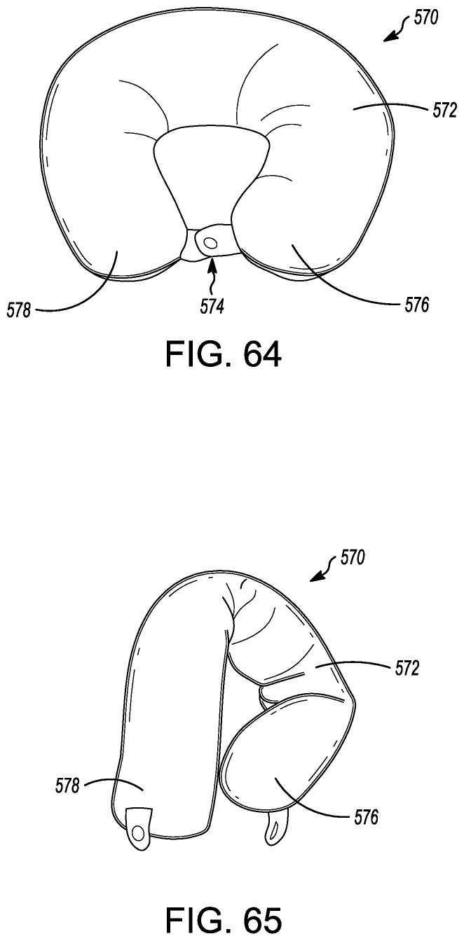

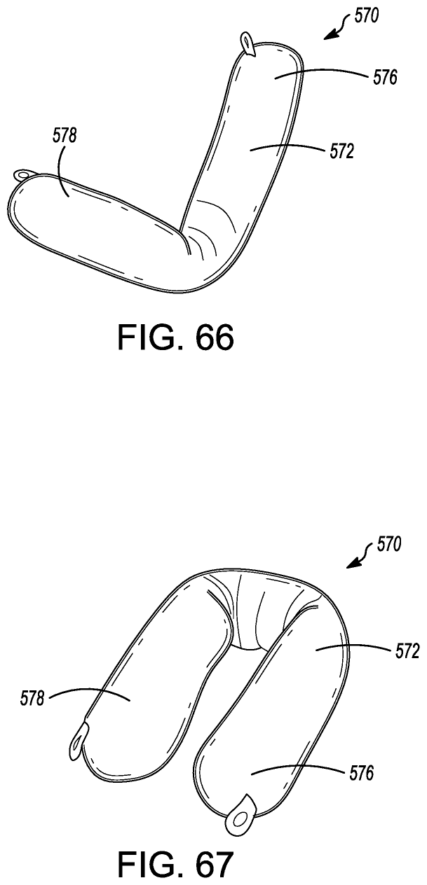

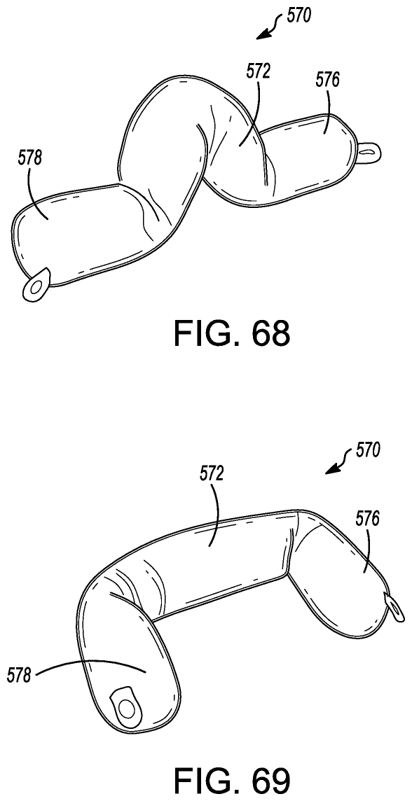

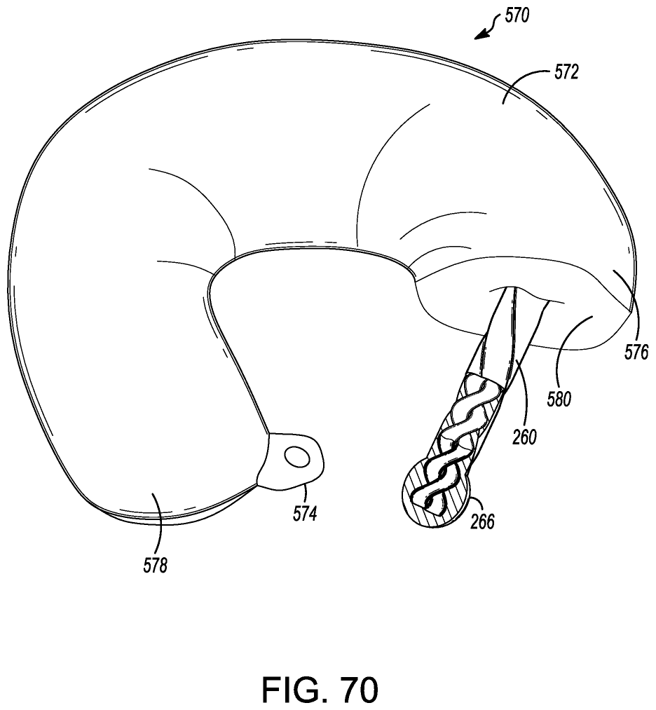

FIGS. 64-70 illustrate a pillow, such as a travel neck pillow 570 having a body 572 with one or more bendable and deformable wires 166. FIG. 64 illustrates the travel neck pillow 570 having a connection means, such as a snap 574 for connecting first and second ends 576, 578 of the body 572 together. Such connection means can be any other means known in the art, such as a buckle, hook and loop fastener and the like. FIGS. 65-69 illustrate the travel neck pillow 570 of FIG. 64 in which the snap 574 is not connected and the body 572 is deformed into various exemplary configurations using the one or more bendable and deformable wires 166. FIG. 70 shows a cut-away to expose an interior of the body 572 having the deformable and bendable wire 166 disposed within a pillow stuffing 580 of the body. In FIG. 70, the bendable and deformable support frame 166 is shown as having the configuration discussed above with regard to twisted wire 260 and ends 266. However, the configuration of wire 166 and/or elongated member 120 can also be used.

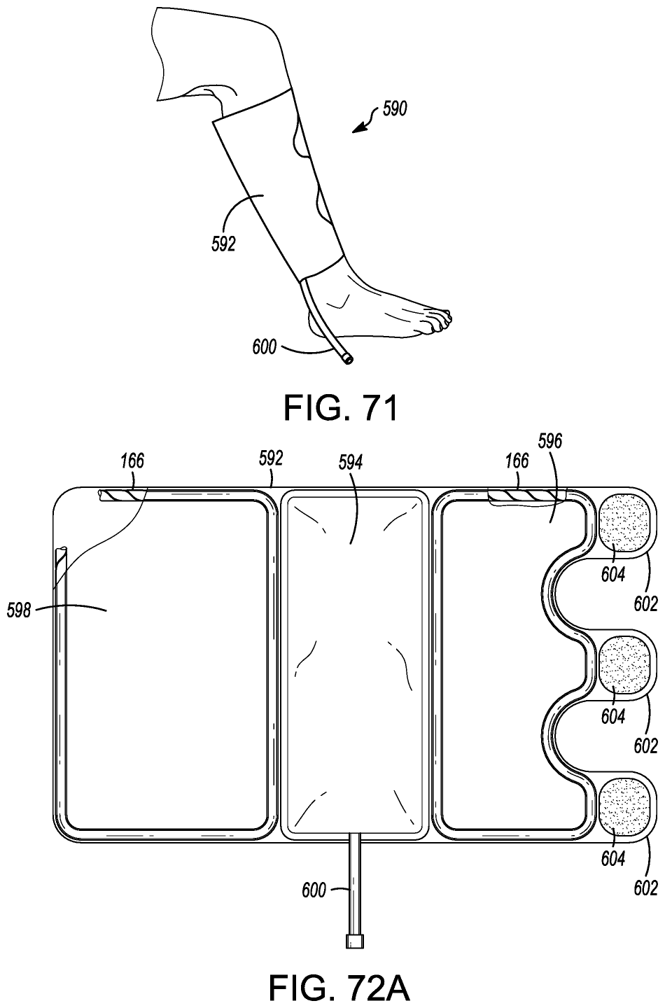

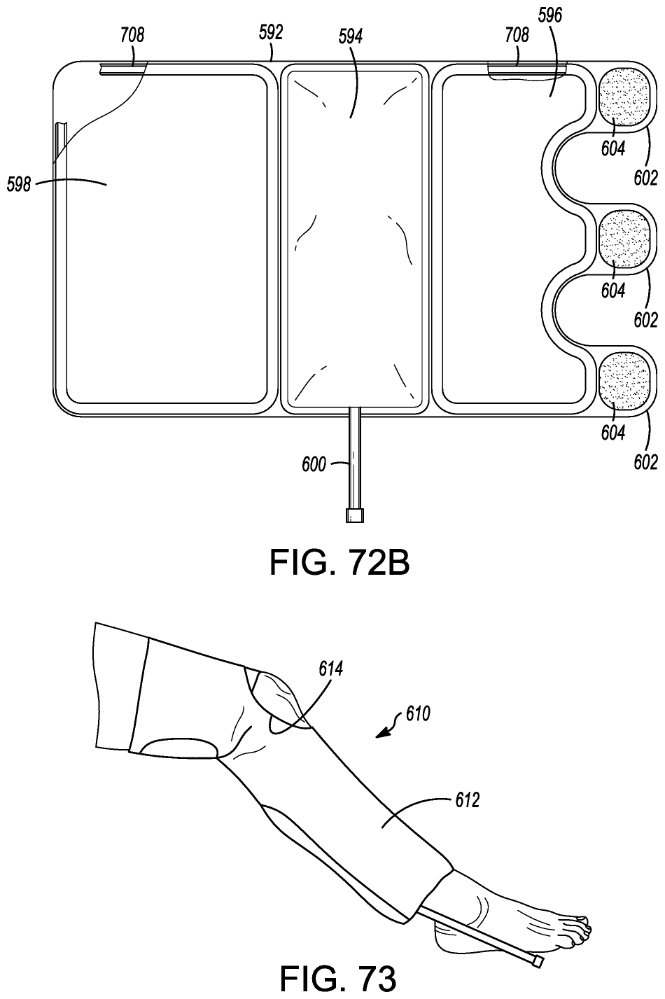

FIG. 71 illustrates a leg compression device 590 having a body 592 with an inflatable bladder 594 for prophylaxis for deep vein thrombosis (DVT) and for use with a DVT system for supplying pressurized fluid to the inflatable bladder 594 and for controlling the amount and/or timing thereof. The body 592 of the leg compression device 590 of FIG. 71 being configured to be wrapped around a lower portion of a patient's leg. The inflatable bladder 594 having an inlet/outlet 600 for supplying and withdrawing the pressurized fluid. FIG. 72a illustrates the leg compression device of FIG. 71 in an unfolded state. As shown in FIG. 72a, the inflatable bladder 594 being positioned between first and second flaps 596, 598 each having the deformable and bendable wire 166 disposed around a periphery of the first and second flaps 596, 598. Although not shown in the configuration of FIG. 72a, the deformable and bendable wire can also be provided around a periphery of the inflatable bladder 594. The body 592 further having securing means for securing the body 592 to the leg of the user, such as straps or strap protrusions 602 projecting from the first flap 596. The protrusions having one portion of a hook and loop fastener 604, where the other portion is provided on the body, such as on the second flap 598.

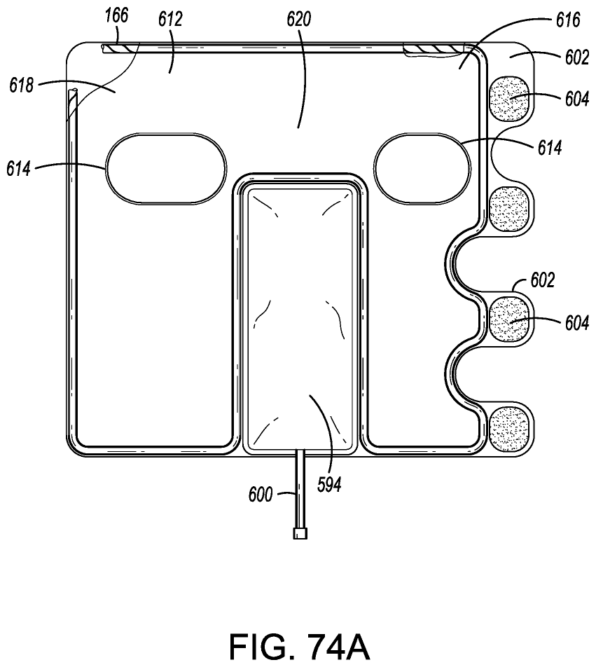

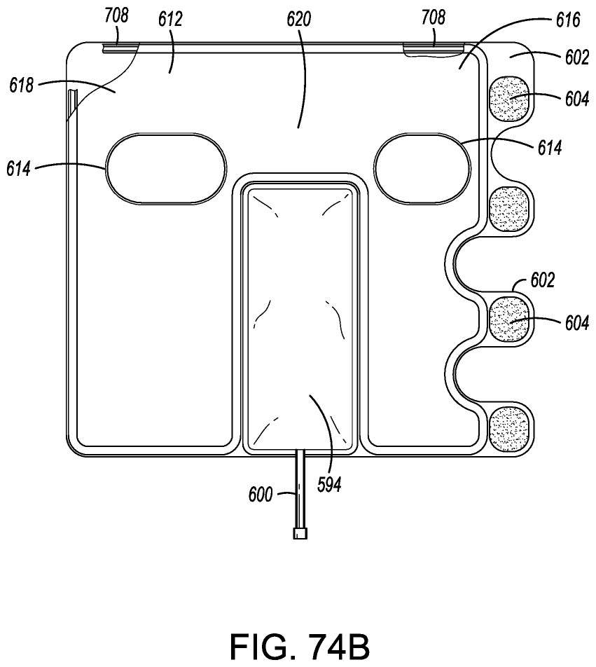

FIG. 73 illustrates a variation of the leg compression device of FIG. 71 where the leg compression device 610 is wrapped around lower and upper portions of a patient's leg (above and below the knee). FIG. 74a illustrates the leg compression device 610 of FIG. 73 in an unfolded state. The body features common to FIGS. 71-74a use common reference numerals. The leg compression device 610 of FIG. 73 being similar to the leg compression device 590 of FIG. 71 except for the first and second flaps 616, 618 being elongated to also cover an upper portion of the leg and the body having knee holes 614 such that the knee can protrude through the knee holes 614 when the body is secured to the upper and lower leg of the user and the knee holes 614 each align over the knee cap. The body 612 also extending in a central portion 620 between the first and second flaps 616, 618. In the configuration of FIGS. 73 and 74a, the deformable and bendable wire 166 extends on the body 612 corresponding to three adjacent sides of the inflatable bladder 594.

In the leg compression devices of FIGS. 71-74a, the bendable and deformable support frame 166 can have the configuration discussed above with regard to twisted wire 260 (with or without ends 266) and/or elongated member 120.

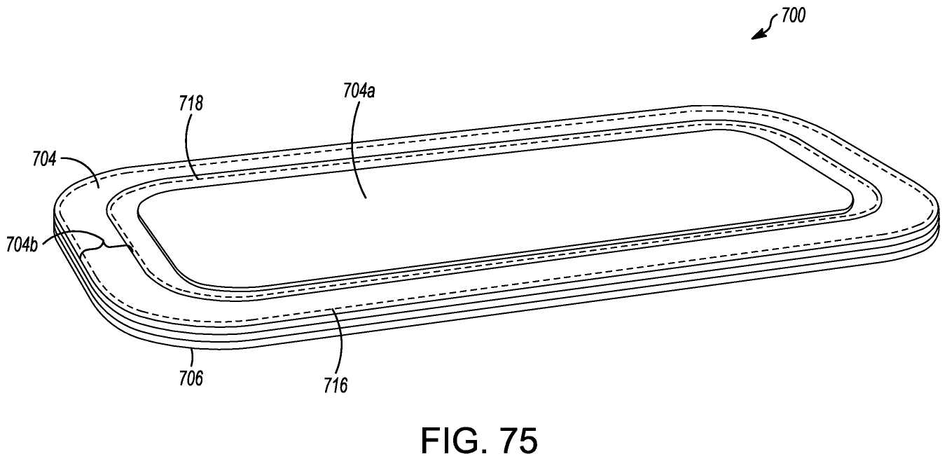

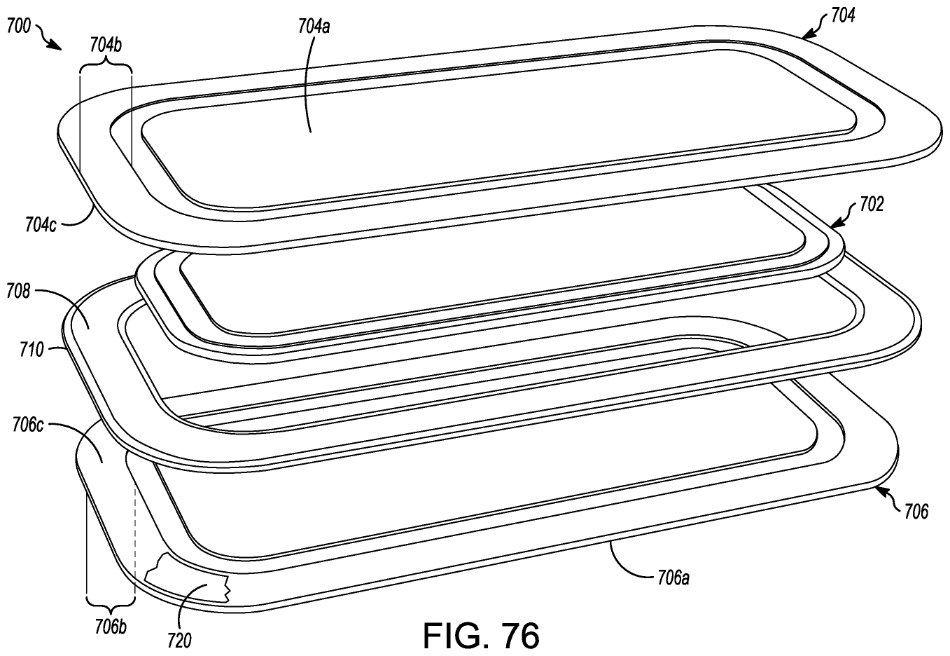

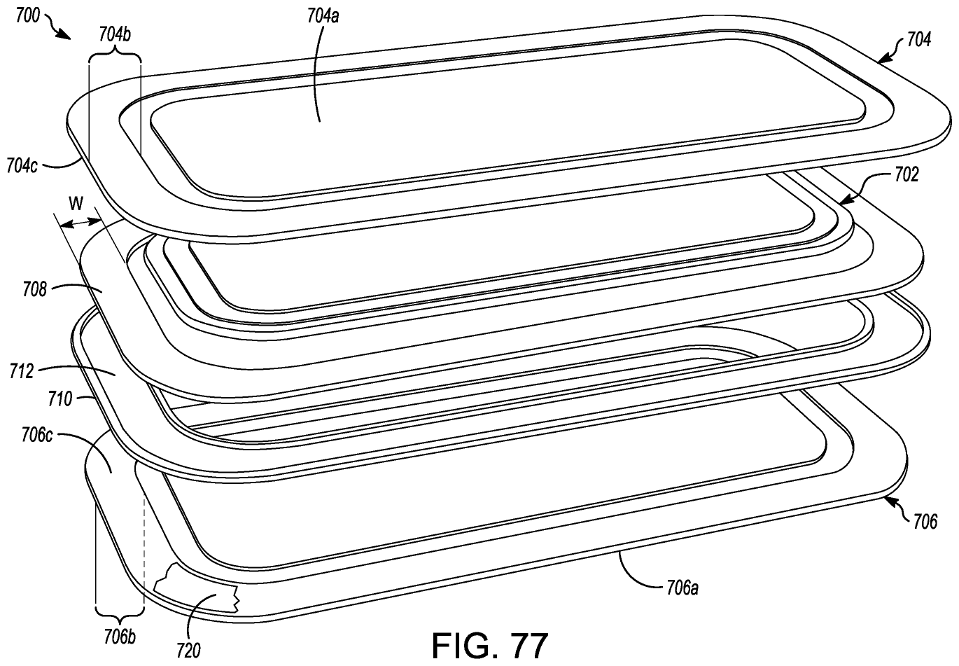

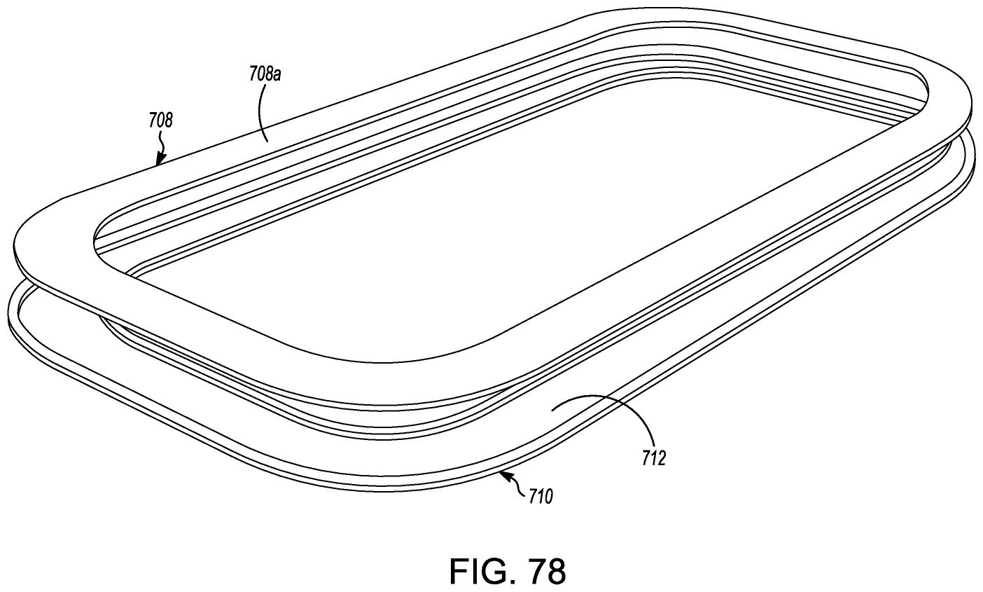

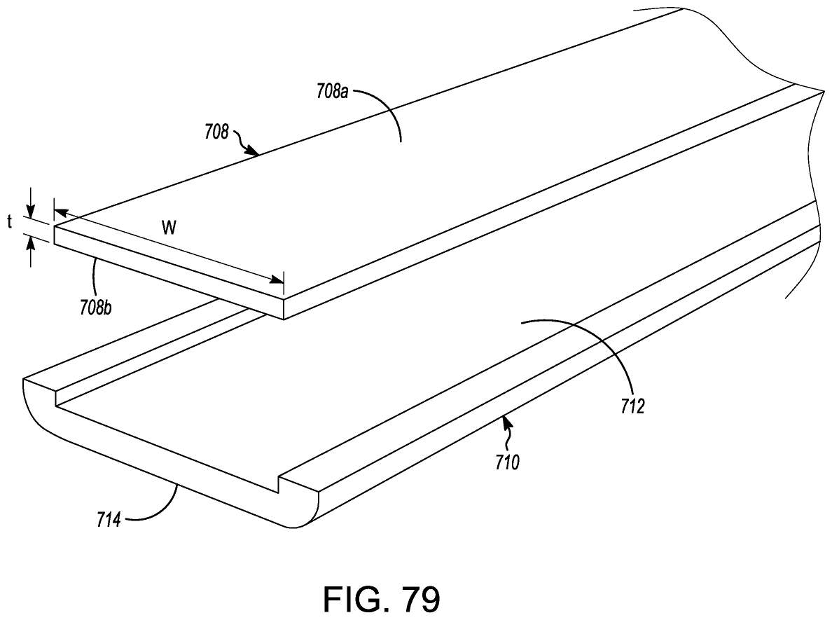

Turning next to FIGS. 75-79, there is shown another embodiment of thermal pack that easily deforms to a shape of body part upon which it is applied and maintains such deformed shape to retain the thermal pack on the body part. Such thermal pack is generally referred to by reference numeral 700. The thermal pack 700 includes a pouch 702 having a thermal material contained with the pouch that can be cooled to apply cold, respectively, to the body part. The pouch 702 can be formed of materials, such as neoprene.

The pouch 702 is sandwiched between top and bottom (or first and second) covers 704, 706, respectively. While all portions of the top and bottom covers 704, 706 can be formed from the same material, such as silicone or canvas (or other woven material), more than one material can be used to form the top and bottom covers 704, 706. For example, the top cover 704 can be a first material, such as canvas (or other woven material) and the bottom cover 506 can be a second material, such as silicone. Furthermore, the top and bottom covers 704, 706 can be formed of a same material, such as silicone, and one or more of an exterior surface of the top and bottom covers 704, 706 can be formed of a different material, which can be the same or different, such as canvas or other woven material. Still further, only an exterior surface field 704a, 706a of the top and bottom covers 704, 706 interiorly of a bonding edge 704b, 706b (discussed below) can be the different material, such as canvas (or other woven material).

The pouch further includes a bendable and deformable support ring 708 formed of a flat sheet and having an approximate width W equal to or slightly less than a corresponding width of the bonding edge 704b, 706b of the top and bottom covers 704, 706, respectively. The support ring 708 can be formed of a metal that can be a “dead soft” alloy, such as aluminum. A thickness t (gauge) of the support ring 708 can be 0.025 inch, 0.032 inch or 0.040 inch.

In a first variation of the thermal pack 700 is assembled by connecting an upper and lower surface 708a, 708b of the support ring 708 to corresponding interior surfaces 704c, 706c of the bonding edges 704b, 706b. Such connection can be made by any means known in the art, such as directly adhering the opposing surfaces (704c with 708a and 706c with 708b) or sewing a portion of an outer edge of the bonding edges 704b, 706b together at an outer portion of the bonding edges 704b, 706b that does not include the support ring 708 therebetween at 716. Additionally, an inner edge of the bonding edges 704b, 706b can be sewn together at an inner portion of the bonding edges 704b, 706b that does not include the support ring 708 therebetween at 718.

With regard to the bonding, methods and adhesives are known in the art for bonding metals to non-metals, such as woven materials and elastomers, such as silicone. In this regard, the interior surfaces 704c, 706c of the bonding edges 704b, 706b can include an adhered transitional material for increasing the bondability with the metal support ring 708. For example, such transitional material can be a woven fabric if the top and bottom covers 704, 706 are formed of silicone or such transitional material can be silicone if the top and bottom covers 704, 706 are formed of a woven material. A portion of the transitional material is shown with broken edges at 720. Although shown with broken edges, such transitional material 720 can extend around one or both of the entire periphery of the interior surfaces 704c, 706c. Methods for bonding metal to silicone are well known in the art and may include, for example, degreasing the surfaces of the metal and/or the silicone or roughening the surfaces of the metal. Other suitable materials for the transitional material include flourosilicone, urethane, neoprene, millathane, EPDM and natural rubber. In additional to bondability, silicone and other similar materials such as those previously listed, maintain a soft durometer at temperatures as low as −100 degrees Fahrenheit.

In a second variation of the thermal pack 700, a gasket 710 is provided for retaining the support ring 708. As shown in FIG. 77-79, the gasket 710 can also be formed in a flat ring shape and have a groove 712 for accepting the support ring 708. The depth of the groove 712 can be approximately equal to the thickness t of the support ring 708. The support ring 708 can loosely fit with the groove 712, fit in an interference manner so as to retain the support ring 708 in the groove 712 or be bonded, such as by adhesive, to the gasket 708. The gasket 710 and support ring 708 assembly can be assembled in the thermal pack 700 similarly as discussed above with regard to the first variation, for example, by bonding and/or sewing.

Additionally, although the second variation illustrates the gasket 710 covering a single (bottom side 708b) of the support ring 708, alternatively, only the top side 708a of the support ring 708 can be covered by the gasket 710, each side 708a, 708b of the support ring 708 can be covered by a gasket (such as the gasket 710 having the groove 712 on one side and another gasket not having a groove or two gaskets each having a groove with a depth ½ of the thickness t of the support ring 708). As another alternative, the gasket can be a single piece with a single opening for insertion of the support ring 708 therein or the metal ring 708 can be insert molded into the gasket 710.

The gasket can have similar properties to the transition material discussed above, such as being formed of silicone, flourosilicone, urethane, neoprene, millathane, EPDM and natural rubber. For example, the gasket 710 can be formed of silicone, which as discussed above is easily adhered to metal, and maintains its soft durometer at very low temperatures. That is, when used in the illustrated configuration, an exterior surface 714 of the gasket 710 is adhered to the inner surface of the corresponding bonding edge, such as inner surface 706c of bonding edge 706b (or transitional material bonded to the inner surface 706c) and a corresponding surface of the support ring 708, such as upper surface 708a is bonded to the inner surface 704c of bonding edge 704b (or transitional material bonded to the inner surface 704c). If each side of the support ring 708 includes the gasket, one side of an exterior surface of the gasket is adhered to the inner surface of the corresponding bonding edge, such as inner surface 706c of bonding edge 706b (or transitional material bonded to the inner surface 706c) and another side of the exterior surface of the gasket is adhered to the inner surface of the corresponding bonding edge, such as inner surface 706c of bonding edge 706b (or transitional material bonded to the inner surface 706c). Additionally or alternatively, as discussed above, the gasket and support ring assembly can be sewn into the thermal pack at the bonding edges 704b, 706b (at inner and/or outer edges of the bonding edges 704b, 706b).

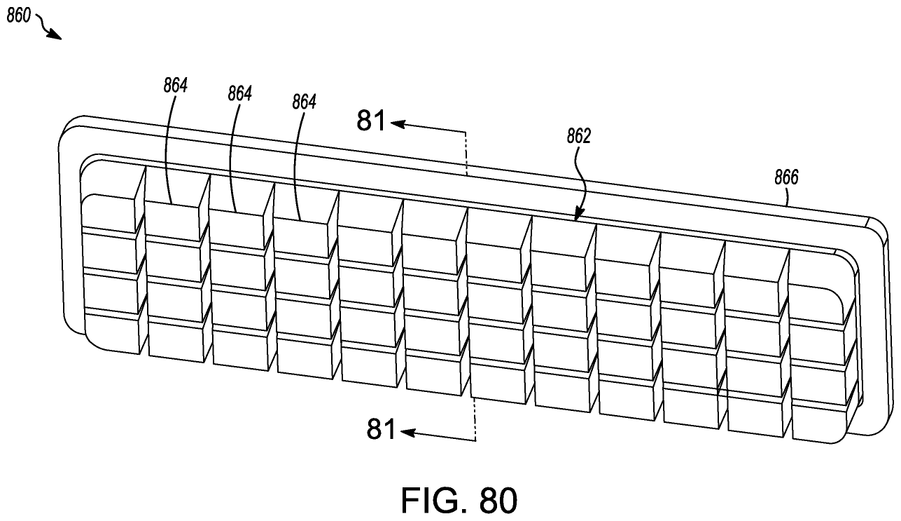

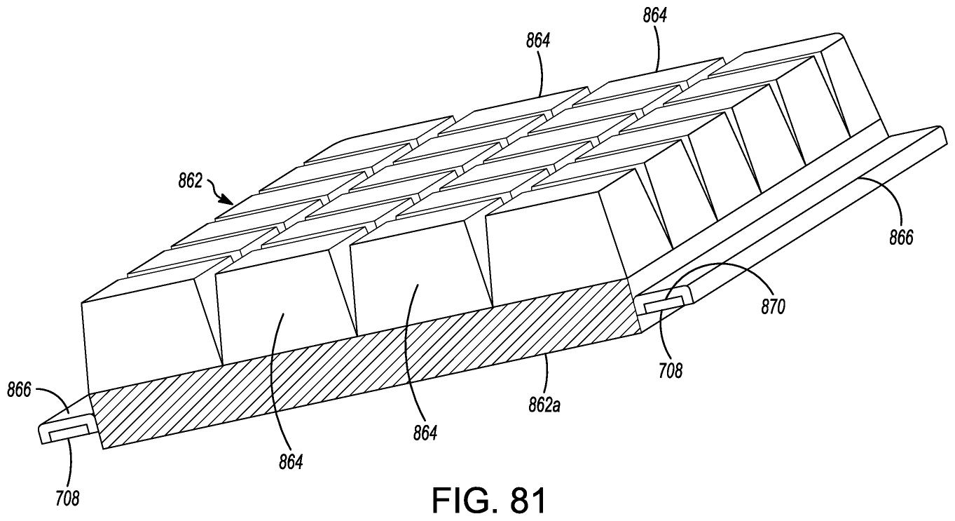

Referring now to FIGS. 80 and 81, there is shown another embodiment of a thermal pack 860 having a segmented pouch 862 formed of a plurality of pouch segments 864. The pouch segments 864 can be any shape or size and provided in any number greater than two. The thermal pack 860 of FIG. 80 illustrates a great number of small pouch segments 864 for increasing flexibility of the pouch and concentrating an amount of thermal material contained in each pouch segment 864.

The thermal pack 860 also includes a bendable and deformable support frame 866 provided at all four sides of the pouch 862 to outline the plurality of pouch segments 864. Male and female connectors can also provided on a periphery of the thermal pack to allow similarly configured thermal packs to be connected together with the thermal pack 860. As shown in FIG. 81, the pouch 862 is formed having the thermal material in both a common portion 862a of the pouch 862 and in each of the pouch segments 864. Furthermore, the bendable and deformable support frame having the metal ring 708 is provided in a cavity 870 apart from the cavity forming the pouch 862 and configured as the flat metal support ring 708. The support frame 866 may comprise the gasket 710 as described above.

Any of the embodiments discussed above can also incorporation the flat metal support ring 708 to replace the wire and/or twisted wire configuration discussed above. For example, the ace bandage of FIGS. 28a, 29a, 31a, 32a can be alternatively configured with a flat metal shaped deformable member 708 as shown in FIGS. 28b, 29b, 31b, 32b, with or without the gasket 710.

Additionally, the thermal packs configured for different parts of the body, for use with a cryotherapy machine and/or for use in a leg compression device, such as those shown in FIGS. 58a, 60a, 72a and 74a can be alternatively configured with a flat metal shaped deformable member 708 as shown in FIGS. 58b, 60b, 72b and 74b with or without the gasket 710.

Since many modifications, variations, and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A thermal pack comprising:
a pouch defining a cavity and having a thermal material disposed within the cavity;
a first cover and a second cover for covering first and second opposing sides, respectively, of the pouch, the first cover and the second cover having a first field portion and a second field portion, respectively, corresponding to the first and second opposing sides, respectively, of the pouch, the first cover and the second cover having a first bonding edge and a second bonding edge, respectively, peripherally outside the first field portion and the second field portion, respectively; and
a bendable and deformable support ring disposed between the first bonding edge and the second bonding edge, the support ring being formed of sheet metal;
wherein the pouch is retained between the first cover and the second cover by one or more of:
at least indirect bonding of a first inner surface of the first bonding edge to a first side surface of the support ring and at least indirect bonding of a second inner surface of the second bonding edge to a second side surface of the support ring; and
sewing one or more of an inner periphery of the first bonding edge to an inner periphery of the second bonding edge and an outer periphery of the first bonding edge to an outer periphery of the second bonding edge.

2. The thermal pack of claim 1, wherein the first cover and the second cover are formed from one or more of canvas and silicone.

3. The thermal pack of claim 1, wherein the support ring is formed from aluminum.

4. The thermal pack of claim 1, wherein the metal ring having a thickness in a range of 0.025 inches to 0.040 inches, inclusive of 0.025 inches and 0.040 inches.

5. The thermal pack of claim 1, further comprising a transitional material disposed on one or more of the first inner surface of the first bonding edge and the second inner surface of the second bonding edge.

6. The thermal pack of claim 1, wherein the transitional material is formed of a material selected from a group consisting of canvas, woven material, silicone, flourosilicone, urethane, neoprene, millathane, EPDM and natural rubber.

7. The thermal pack of claim 1, further comprising a gasket for covering one or more of the first side surface and the second side surface of the support ring.

8. The thermal pack of claim 7, wherein the gasket is formed of a material selected from a group consisting of silicone, flourosilicone, urethane, neoprene, millathane, EPDM and natural rubber.

9. An elastic bandage comprising:
a body formed of a typical stretchable material; and
one or more bendable and deformable support rings disposed at least along a portion of a length of the body, the one or more support rings being configured such that the body is deformed into a shape matching a contour of a body part and the body is maintained in the shape while applied to the body part by the one or more supports, the one or more support rings being formed of sheet metal.

10. The elastic bandage of claim 9, wherein the one or more bendable and deformable support rings are disposed along an entire length of the body.

11. The elastic bandage of claim 9, wherein the one or more bendable and deformable support rings are disposed on a surface of the body.

12. The elastic bandage of claim 11, further comprising a covering disposed on the surface of the body to cover the one or more bendable and deformable support rings.

13. The elastic bandage of claim 9, wherein the body comprises first and second layers and the one or more bendable and deformable support rings are disposed between the first and second layers.

14. The elastic bandage of claim 13, wherein the first and second layers comprise a tubular sleeve.

15. The elastic bandage of claim 9, wherein the body comprises:
a hole configured to accept a thumb of a hand of the user; and
one or more straps configured to wrap the body around the hand of the user.

16. The elastic bandage of claim 15, wherein the one or more bendable and deformable support rings comprising a single bendable and deformable support ring disposed around a periphery of the body.

17. A leg compression device for use with a device for providing compressed fluid, the leg compression device comprising:
a body having an inflatable bladder, the inflatable bladder being inflated upon delivery of the compressed fluid to the inflatable bladder;
a bendable and deformable support ring one of attached to or disposed within the body, the support being configured such that the body is deformed into a shape matching a contour of a body part and the body is maintained in the shape while applied to the body part by the support, the support ring being formed of sheet metal; and
tubing having one end in fluid communication with the cavity and another end in fluid communication with the device such that the device provides the compressed fluid to inflatable bladder via the tubing.

18. The leg compression device of claim 17, wherein the body comprises:
first and second flaps each extending from the inflatable bladder; and
one or more securing straps for securing the body to the body part of the user.

19. The leg compression device of claim 18, wherein the support ring is provided on a periphery of each of the first and second flaps of the body.
